(19) United States
(12) Patent Application Publication
(10) Pub. No.: US 2011/0212133 A1
(43) Pub. Date: Sep. 1, 2011

(54) Novel Methods of Diagnosis and Treatment of P. Aeruginosa Infection and Reagents Therefor

(76) Inventors: Andrew John Sloane, Balmain (AU); Susanne Kartin Pedersen, North Ryde (AU); Ron Weinberger, North Bondi (AU)

(21) Appl. No.: 13/020,639

(22) Filed: Feb. 3, 2011

Related U.S. Application Data
(63) Continuation of application No. 11/571,036, filed on Jan. 21, 2009, now abandoned.

(30) Foreign Application Priority Data
Jun. 28, 2004 (AU) 2004903521

(51) Int. Cl. A61K 39/104; G01N 33/566; G01N 33/577; G01N 33/573; C07K 16/12; C07K 16/40; C12N 9/12; C12N 9/02; C07K 2/00; A61P 37/04; A61P 31/04

(52) U.S. Cl. 424/260.1; 436/501; 435/7.1; 530/389.5; 435/194; 435/189; 530/350

(57) ABSTRACT

The present invention relates to novel diagnostic, prognostic and therapeutic reagents for infection of an animal subject such as a human by Pseudomonas aeruginosa, and conditions associated with such infections, such as, for example, an acute clinical exacerbation in a cystic fibrosis (CF) subject. In particular, the present invention relates to methods for diagnosing/prognosing an infection by P. aeruginosa in a subject comprising detecting the presence or amount of one or more proteins of P. aeruginosa or a fragment or epitope thereof or an antibody thereto in a sample from the subject.

### CROSS REFERENCE TO RELATED APPLICATIONS

This application is a continuation of co-pending U.S. application Ser. No. 11/571,036, filed Dec. 20, 2006, which is a National Stage of International Application No. PCT/AU2005/000942, filed Jun. 28, 2005, and claims the right of priority based on Australian application serial no. 2004903521, filed Jun. 28, 2004, which are all herein incorporated in their entirety by reference.

### FIELD OF THE INVENTION

The present invention relates to novel diagnostic, prognostic and therapeutic reagents for infection of an animal subject such as a human by P. aeruginosa, and conditions associated with such infections, such as, for example, an acute clinical exacerbation in a cystic fibrosis (CF) subject.

### BACKGROUND OF THE INVENTION

1. General Information

This specification contains nucleotide and amino acid sequence information prepared using PatentIn Version 3.3, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, proteomics, virology, recombining DNA technology, peptide synthesis in solution, solid phase peptide synthesis, and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Immobilized Cells and Enzymes: A Practical Approach (1986) IRL Press, Oxford, whole of text;
6. Perbal, B., A Practical Guide to Molecular Cloning (1984);
7. Methods In Enzymology (S. Colowick and N. Kaplan, eds., Academic Press, Inc.), whole of series;
8. J. F. Ramalho Ortigão, “The Chemistry of Peptide Synthesis” In: Knowledge database of Access to Virtual Laboratory website (Interactiva, Germany);
9. Sakakibara, D., Teichman, J., Lien, E. Land Fenichel, R. L. (1976). Biochem. Biophys. Res. Commun. 73 336-342
10. Merrifield, R. B. (1963). J. Am. Chem. Soc. 85, 2149-2154.
11. Barany, G. and Merrifield, R. B. (1979) in The Peptides (Gross, E. and Meienhofer, J. eds.), vol. 2, pp. 1-284, Academic Press, New York.
12. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
13. Bodanszky, M. (1984) Principles of Peptide Synthesis, Springer-Verlag, Heidelberg.
14. Bodanszky, M. & Bodanszky, A. (1984) The Practice of Peptide Synthesis, Springer-Verlag, Heidelberg.
15. Bodanszky, M. (1985) Int. J. Peptide Protein Res. 25, 449-474.
16. Handbook of Experimental Immunology, Vols. I-IV (D. M. Weir and C. C. Blackwell, eds., 1986, Blackwell Scientific Publications).
17. Wilkins M. R., Williams K. L., Appel R. D. and Hochstrasser (Eds) 1997 Proteome Research: New Frontiers in Functional Genomics Springer, Berlin.

2. Description of the Related Art

Pseudomonas aeruginosa

Pseudomonas aeruginosa (P. aeruginosa) is an aerobic, motile, gram-negative, rod. P. aeruginosa inhabits soil, water and vegetation in nature. While this bacterium seldom causes disease in healthy people, P. aeruginosa is an opportunistic pathogen which accounts for approximately 10% of all nosocomial infections (National Nosocomial Infection Survey report-Data Summary from October 1986-April 1996). In fact, P. aeruginosa is the fourth most commonly isolated nosocomial pathogen accounting for 10.1 percent of all hospital-acquired infections. P. aeruginosa is the most common pathogen affecting Cystic Fibrosis (CF) patients with 61% of the specimens culturing positive (Govan and Deretic, Microbiol. Reviews, 60:530-574, 1996). Additionally, P. aeruginosa is one of the two most common pathogens observed in intensive care units (ICUs) (Jarvis, W. R. et al., 1992, J. Antimicrob. Chemother., 29(a supp.):19-24). Mortality rates from P. aeruginosa infections have been observed as high as 50%.

P. aeruginosa infection is associated with urinary tract infections, respiratory system infections, dermatitis, soft tissue infections, bacteremia, bone and joint infections, gastrointestinal infections and a variety of systemic infections. Patients suffering from severe burns, cancer, AIDS patients, cystic fibrosis or who are immunosuppressed are particularly susceptible to P. aeruginosa infection.

Presently, P. aeruginosa infection can be effectively controlled by antibiotics, particularly using a combination of drugs. However, resistance to several of the common antibiotics has been shown and is particularly problematic in ICUs (Archibald et al., Clin. Infectious Dis., 24:211-215, 1997; Fish, et al., Pharmacotherapy, 15:279-291, 1995). In addition, P. aeruginosa has already demonstrated mechanisms for acquiring plasmids containing antibiotic resistance genes (Jakoby, G. A. (1986), The bacteria, Vol. X, The biology of Pseudomonas, pp. 265-294, J. R. Sokach (ed.) Academic Press, London) and at present there are no approved vaccines for Pseudomonas infection.

Currently, the diagnosis of a P. aeruginosa infection is based upon clinical findings, microbiological cultures and biochemical tests. Gram stains of direct smears from patients are of little or no value. In fact, the most reliable method to date is the isolation of the bacterium in pure culture and its subsequent identification by biochemical or serological methods. Conventional laboratory culture techniques involve incubating clinical samples for between about 24 to about 48 hours to allow the organisms to multiply to macroscopically detectable levels. Subculture techniques and metabolic assays are then required to distinguish P. aeruginosa from related pseudomonads and other enteric bacteria and may require an additional 24 to 48 hours. Accordingly, while the rapid and accurate diagnosis of a P. aeruginosa infection is highly desirable, it is not currently possible using existing reagents and techniques.

In an effort to expedite and simplify the diagnostic process for the often life-threatening infections caused by P. aeruginosa, several immunological approaches have been attempted. For example, immunofluorescent detection of P. aeruginosa using polyclonal antisera produced in rabbits is described in Ajello et al., Invest. Urology, 5:203 (1967); Sands et al., J. Clin. Path., 28:997 (1975); and Kohler et al., J. Clin. Microbiol., 9:253 (1979). However, as a result of a variety of problems inherent in such preparations, these reagents have not found acceptance in clinical laboratories.

Monoclonal antibodies have been produced against several components of lipopolysaccharide (LPS) of P. aeruginosa. However, monoclonal antibodies that bind to the O-side chains of LPS are generally serotype or immunotype specific and are incapable of detecting all serotypes or immunotypes of P. aeruginosa. Monoclonal antibodies that bind to the core and/or lipid A portions of LPS are non-specific and are capable of also binding to LPS from other species of Pseudomonas or other gram-negative bacteria.

Accordingly, it is clear that there is a need in the art for a rapid and reliable assay for detecting an infection by P. aeruginosa in a subject.

### Cystic Fibrosis

Cystic fibrosis (CF) is one of the most common fatal autosomal recessive diseases affecting Caucasian populations. CF has an incidence in neonatals of about 0.05%, indicating a carrier frequency of about 5% of the population. Biological parents of subjects with CF are, by definition, obligatory carriers. Carriers are clinically normal and their detection prior to the birth of an affected child has been precluded by the absence of detectable effects of the gene in single dose.

CF is a disease of the exocrine glands, affecting most characteristically the pancreas, respiratory system, and sweat glands. The disease usually begins during infancy and the prognosis for an affected child with CF is a median life expectancy currently estimated to be 30 years.

CF is typified by chronic respiratory infection, pancreatic insufficiency, and susceptibility to heat prostration. It is a major cause of death in children. It is estimated that there are between ten million and twelve million carriers for cystic fibrosis in the United States. Each year, between two thousand and three thousand children are born in the United States who are affected by cystic fibrosis. The cost of therapy for cystic fibrosis patients can exceed US$20,000 per year per patient. Of patients diagnosed in early childhood, fewer than fifty percent reach adulthood.

A serious consequence of CF is an exacerbated clinical condition or exacerbated state. As used herein the term “acute clinical exacerbation”, “acute exacerbation”, “clinical exacerbation”, “exacerbation”, or “exacerbated state” in the context of a CF patient shall be understood to mean an exaggeration of a pulmonary symptom of CF.

In most cases, such a clinical exacerbation will be a consequence of a respiratory infection and/or increased inflammation. The term “respiratory infection” in this context includes invasion by and/or multiplication and/or colonisation of a pathogenic microorganism in one or more components of the respiratory tract, such as, for example, lung, epiglottis, trachea, bronchi, bronchioles, or alveoli. Commonly, such infections result in the inflammation of the respiratory tract.

CF patients are particularly susceptible to respiratory infections from bacteria, and, in particular, P. aeruginosa. For example a chronic respiratory infection, particularly an infection of the lung by P. aeruginosa, accounts for almost 90% of the morbidity and mortality in CF. By age 12, about 60-90% of CF patients are infected with P. aeruginosa.

Progressive loss of pulmonary function over many years due to chronic infection with mucoid P. aeruginosa is common in subjects suffering from CF. Smith et al., Cell. 85, 229-236, 1996, reported defective bacterial killing by fluid obtained from airway epithelial cell cultures of CF patients, and suggested that this phenomenon was due to the inhibition of an unidentified antimicrobial factor resulting from increased levels of sodium chloride in the airway epithelial fluid.

Severe chronic pulmonary disease is also associated with cases of CF wherein CFTR expression on the cell surface is reduced, such as, for example, in patients carrying the −F508 mutation. Pier et al. Science. 271, 64-67, 1996 proposed that ingestion and clearance of P. aeruginosa by epithelial cells may protect the lungs against infection, since the specific ingestion and clearance of P. aeruginosa was compromised in a cell line derived from a patient with the ΔF508 mutation.

Patients suffering from CF are extremely susceptible to acute clinical exacerbations, often resulting in a further increase in inflammation and mucus production, thus increasing the risk of bronchiectasis and eventually respiratory failure.

An acute clinical exacerbation is generally assessed using the protocols described in Williams et at Australian Journal of Physiotherapy, 47, 227-236, 2001; Dakin et al, Pediatr Pulmonol 34, 436-442, 2001; and/or Rosenfeld et al, J. Pediatr 139 359-365, 2001. In particular, several criteria are assessed, and a patient satisfying four or more of these criteria is considered to have an acute clinical exacerbation. These criteria are as follows:
i. Change in sputum production (volume, colour, consistency);
ii. New or increased haemoptysis;
iii. Increased cough;
iv. Increased dyspnoea (shortness of breath);
v. Malaise, fatigue or lethargy;
vi. Decreased exercise tolerance;
vii. Fever;
viii. Anorexia or weight loss;
ix. Sinus pain/tenderness or change in sinus discharge;
x. FVC or FEV₁ decreased 10% from previous recorded value;
xi. Radiographic changes indicative of a pulmonary infection; and
xii. Changes in chest sounds.

Clearly, these methods are subjective and, as a consequence, subject to human error potentially leading to either over-diagnosis or under-diagnosis of an acute clinical exacerbation.

Alternatively, an acute clinical exacerbation is diagnosed by detecting the concentration of C-reactive protein, determining erythrocyte sedimentation rate and/or peripheral neutrophil counts as reviewed in H\ddot{n}er et al, Med Bull Istanbul, 32(1), 1999. However, these assays rely upon the detection of human proteins or cells, the level or number of which are modulated by a variety of factors in addition to an acute pulmonary exacerbation.

Whilst there has been significant progress in diagnosing CF, the need still exists for further diagnostic and prognostic assays for complications arising in patients suffering from the disease, in particular rapid and reliable methods for determining whether or not a subject suffering from CF at risk of developing or is developing or is recovering from an acute clinical exacerbation. Clearly, an assay that diagnoses an infection by P. aeruginosa will provide such a diagnostic/prognostic assay.

### SUMMARY OF INVENTION

In work leading up to the present invention, the inventors sought to isolate and identify proteins from P. aeruginosa to which a subject suffering from a P. aeruginosa infection raises or has raised an immune response.

The inventors used an immunocapture approach to identify P. aeruginosa proteins against which subject infected with said bacterium had raised an immune response, and, in particular, an antibody response. A number of immunogenic P. aeruginosa proteins were identified in vivo in samples derived from P. aeruginosa-infected patients. Furthermore, the present inventors have identified subjects suffering from a P. aeruginosa infection using several of the identified proteins.

The present inventors have shown that a subject suffering from a P. aeruginosa infection raises specific antibodies to a protein selected from the group consisting of ferric iron-binding protein (HitA), thioredoxin dependent reductase (PAPS), thioredoxin, heat shock protein GroES, nucleotide dependent kinase (NDK) and DNA-binding protein HU. Furthermore, the inventors showed that each of these proteins were present in a biological sample of an infected subject (i.e. that each protein is expressed in vivo in a subject suffering from a P. aeruginosa infection).

Without limiting the present invention, several of the identified proteins are stress proteins of P. aeruginosa and/or associated with growth of P. aeruginosa under, for example, anaerobic growth of P. aeruginosa, however may also be found in aerobically grown P. aeruginosa. For example, ferric iron-binding protein (HitA), thioredoxin, heat shock protein GroES, and DNA-binding protein HU.

Some of the proteins are also or alternatively involved in alginate syntheses by P. aeruginosa, in particular, nucleotide dependent kinase (NDK) and thioredoxin dependent reductase (PAPS).

With particular regard to NDK, the present inventors also showed that the detected protein was also phosphorylated. This form of NDK is considered to have a role in extracellular alginate synthesis, a virulence factor of P. aeruginosa. Such modified proteins are encompassed by the present invention and, in particular, by the term “a protein associated with anaerobic growth of P. aeruginosa”.

These findings have provided the means for producing novel diagnostics for the detection of P. aeruginosa infection in a subject, and novel prognostic indicators for the progression of infection or a disease state associated therewith. Preferably, a marker referred to supra or antibodies thereto are useful for the early diagnosis of infection or disease. It will also be apparent to the skilled person that such prognostic indicators as described herein may be used in conjunction with therapeutic treatments for P. aeruginosa infection.

Accordingly, the present invention provides a method for diagnosing an infection by P. aeruginosa and/or an acute clinical exacerbation in a subject comprising detecting in a biological sample from said subject a protein of P. aeruginosa or an immunogenic fragment or epitope thereof, wherein the presence of said protein in the sample is indicative of infection and/or exacerbation.

The present invention additionally provides a method for diagnosing an infection by P. aeruginosa or an acute clinical exacerbation in a subject comprising detecting in a biological sample from said subject antibodies against a protein of P. aeruginosa or an immunogenic fragment or epitope thereof, wherein the presence of said antibodies in the sample is indicative of infection. The infection may be a past or present infection, or a latent infection.

In one embodiment, a protein of P. aeruginosa is a protein that is upregulated or expressed when said bacterium is grown under anaerobic conditions, e.g. in a host and/or a protein that is involved in extracellular alginate synthesis in or by P. aeruginosa. Clearly, such a protein may also be expressed or upregulated under other conditions, in particular, aerobic growth conditions and/or in response to stress. As exemplified herein, a protein of P. aeruginosa detected in an infected subject is selected from the group consisting of HitA, PAPS, thioredoxin, GroES, NDK and DNA binding protein HU.

In a preferred embodiment, a protein of P. aeruginosa is selected from the group consisting of:
(i) ferric iron-binding protein (HitA) comprising an amino acid sequence at least 80% identical to the amino acid sequence set forth in SEQ ID NO: 1;
(ii) thioredoxin dependent reductase (PAPS) comprising an amino acid sequence at least 80% identical to the amino acid sequence set forth in SEQ ID NO: 2;
(iii) thioredoxin comprising an amino acid sequence at least 80% identical to the amino acid sequence set forth in SEQ ID NO: 3;
(iv) heat shock protein GroES comprising an amino acid sequence at least 80% identical to the amino acid sequence set forth in SEQ ID NO: 4;
(v) nucleotide dependent kinase (NDK) comprising an amino acid sequence at least 80% identical to the amino acid sequence set forth in SEQ ID NO: 5;
(vi) DNA-binding protein HU comprising an amino acid sequence at least 80% identical to the amino acid sequence set forth in SEQ ID NO: 6; and
(vii) mixtures thereof.

The present invention also encompasses detection of a modified form of a protein of P. aeruginosa (e.g. a protein described supra), such as, for example, a phosphorylated protein, a glycosylated protein, a lipitated protein or an antibody that binds thereto.

As used herein, the term “infection” shall be understood to mean invasion and/or colonisation by a microorganism and/or multiplication of a micro-organism, in particular, a bacterium or a virus, a subject. Such an infection may be unapparent or result in local cellular injury. The infection may be localised, subclinical and temporary or alternatively may spread by extension to become an acute or chronic clinical infection. The infection may also be a past infection wherein residual antigen from a protein associated with anaerobic growth of P. aeruginosa, or alternatively, reactive host antibodies that bind to isolated from a protein of P. aeruginosa protein or peptides therefrom, remain in the host. The infection may also be a latent infection, in which the microorganism is present in a subject, however the subject does not exhibit symptoms of disease associated with the organism. Preferably, the infection is a respiratory infection by P. aeruginosa. However, the term infection also encompasses a P. aeruginosa infection of a wound (e.g. a burn), an infection of the meninges (e.g. meningitis), a urinary tract infection, an infection of a heart valve (e.g. endocarditis), an ear infection, an eye infection, a bone infection (e.g., Vertebral osteomyelitis), a skin infection or a gastro-intestinal infection.

As used herein the term “respiratory tract” shall be taken to mean a system of cells and organs functioning in respiration, in particular the organs, tissues and cells of the respiratory tract include, lungs, nose, nasal passage, paranasal sinuses, nasopharynx, larynx, trachea, bronchi, bronchioles, respiratory bronchioles, alveolar ducts, alveolar sacs, alveoli, pneumocytes (type 1 and type 2), ciliated mucosal epithelium, mucosal epithelium, squamous epithelial cells, mast cells, goblet cells, and intraepithelial dendritic cells.

The present invention also provides a method for determining the progression of a P. aeruginosa infection or an acute clinical exacerbation in a subject being administered with an amount of a therapeutic compound for the treatment of said infection or exacerbation, said method comprising detecting in a biological sample from the subject a protein of P. aeruginosa or mixtures thereof wherein the presence of said protein indicates that the subject has not recovered from the infection or exacerbation. In accordance with this embodiment, a level of the protein of P. aeruginosa that is less than a level of that protein detectable in a subject suffering from an acute clinical exacerbation indicates that the subject is recovering from an exacerbated state.

The present invention also provides a method for determining the progression of a P. aeruginosa infection or an acute clinical exacerbation in a subject being administered with an amount of a therapeutic compound for the treatment of said infection or exacerbation, said method comprising detecting in a biological sample from the subject an antibody against a protein of P. aeruginosa or mixtures thereof wherein the presence of said antibody indicates that the subject has not recovered from the infection or exacerbation. In accordance with this embodiment, a level of the antibody against a protein of P. aeruginosa that is less than a level of that protein detectable in a subject suffering from an acute clinical exacerbation indicates that the subject is recovering from an exacerbated state.

The present invention also provides a method of treatment of a P. aeruginosa infection or an acute pulmonary exacerbation in a subject comprising performing a diagnostic method or prognostic method as described herein. In one embodiment, the present invention provides a method of treatment comprising:
(i) detecting the presence of P. aeruginosa infection in a biological sample from a subject; and
(ii) administering a therapeutically effective amount of a pharmaceutical composition to reduce the number of pathogenic bacterium in the lung, blood or lymph system of the subject.

As the presence of a protein of P. aeruginosa in a subject elicits a specific antibody response against said protein, the invention additionally provides a method of eliciting the production of an antibody against P. aeruginosa comprising administering an isolated protein of P. aeruginosa or an immunogenic fragment or epitope thereof to said subject for a time and under conditions sufficient to elicit the production of antibodies, such as, for example, neutralizing antibodies against P. aeruginosa.

The present invention clearly contemplates the use of a protein of P. aeruginosa or an immunogenic fragment or epitope thereof in the preparation of a therapeutic or prophylactic subunit vaccine against P. aeruginosa infection in a human or other animal subject.

Accordingly, the invention also provides a vaccine comprising a protein of P. aeruginosa or an immunogenic fragment or epitope thereof in combination with a pharmaceutically acceptable diluent. Preferably, the protein or epitope thereof is formulated with a suitable adjuvant.

Alternatively, the peptide or derivative or variant is formulated as a cellular vaccine via the administration of an autologous or allogeneic antigen presenting cell (APC) or a dendritic cell that has been treated in vitro so as to present the peptide on its surface.

Nucleic acid-based vaccines that comprise nucleic acid, such as, for example, DNA or RNA, encoding the immunologically active protein of P. aeruginosa or epitope(s) and cloned into a suitable vector (e.g. vaccinia, canary pox, adenovirus, or other eukaryotic virus vector) are also contemplated. Preferably, DNA encoding a protein of P. aeruginosa is formulated into a DNA vaccine, such as, for example, in combination with the existing Calmette-Guerin (BCG) or an immune adjuvant such as vaccinia virus, Freund's adjuvant or another immune stimulant.

The present invention further provides for the use of an isolated protein of P. aeruginosa or an immunogenic fragment or epitope thereof in the preparation of a composition for the prophylactic or therapeutic treatment or diagnosis of infection by P. aeruginosa in a subject.

The present invention additionally provides a kit for detecting P. aeruginosa infection in a biological sample. In one embodiment, the kit comprises:
(i) one or more isolated antibodies that bind to a protein of P. aeruginosa or an immunogenic fragment or epitope thereof; and
(ii) means for detecting the formation of an antigen-antibody complex.

In an alternative embodiment, the kit comprises:
(i) an isolated or recombinant protein of P. aeruginosa or an immunogenic fragment or epitope thereof and
(ii) means for detecting the formation of an antigen-antibody complex.

As used herein, the term “protein of P. aeruginosa” shall preferably refer to a protein selected from the group consisting of Ferric iron-binding protein (HitA), thioredoxin dependent reductase (PAPS), thioredoxin, GroES, nucleotide dependent kinase (NDK) and DNA-binding protein HU or mixtures thereof.

### BRIEF DESCRIPTION OF THE DRAWINGS

FIG. 1A is a photographic representation showing proteins from P. aeruginosa that have been separated using two-dimensional gel electrophoresis and probed with serum from a non-CF healthy control subject. Antibody binding is detected using chemiluminescence.

FIG. 1B is a photographic representation showing proteins from P. aeruginosa that have been separated by two-dimensional gel electrophoresis and probed with serum from a CF subject that suffers from a P. aeruginosa infection. Antibody binding is detected using chemiluminescence.

FIG. 2 is a photographic representation showing a 2-dimensional gel showing proteins that have been captured from P. aeruginosa using an immunoglobulin-containing fraction from a plurality of CF subjects suffering from a P. aeruginosa infection.

FIG. 3A is a graphical representation of a full-scan MALDI MS spectrum showing peptide masses collected from the tryptic digested spot 7 (FIG. 2). Peptide masses matching to tryptic fragments of NDK are marked by arrows. The insert shows an enlarged view of the 1426 m/z peptide.

FIG. 3B is a graphical representation showing results of MALDI-MS analysis of a phosphatase treated tryptic digest of NDK resulting in collection of 13 specific peptides (75% coverage). A dominating 1346.7 m/z peptide (matching the oxidised tryptic peptide (theoretical) from amino acid 34 to amino acid 45 of NDK) was obtained. This peptide was not observed in non-phosphatase treated samples (FIG. 3A). This peptide appears to be a cognate phosphopeptide of the tryptic 1426 m/z peptide (34-45) of NDK.

FIG. 3C is a graphical representation showing results of MALDI-MS post-source decay fragmentation analysis of sulfonated, phosphatase treated 1346.7 m/z peptide (FIG. 3B). The resulting sulfonated peptide had a mass of 1560.8 m/z. Insert shows full scan MALDI-MS spectrum of the sulfonated, phosphatase treated tryptic NDK digest.

FIG. 4A is a photographic representation showing immunoreactivity of four CF subject and three healthy control subjects to P. aeruginosa proteins HitA, thioredoxin, GroES and NDK. Each spot position in the 4- or 5-spot containing grid shows the immunoreactivity of a single subject to the protein onto which plasma aliquots were analysed. Spot positions 1 to 3 are from healthy control subjects. Spot positions 4 to 7 are from CF subjects.

FIG. 4B is a photographic representation showing immunoreactivity of four CF subject and three healthy control subjects to PBS or BSA (i.e., negative controls for the experiment shown in FIG. 4A). Each spot position in the 4- or 5-spot containing grid shows the immunoreactivity of a single subject to the protein onto which plasma aliquots were analysed. Spot positions 1 to 3 are from healthy control subjects. Spot positions 4 to 7 are from CF subjects.

### DETAILED DESCRIPTION OF THE PREFERRED EMBODIMENTS

An Enhanced Level of Protein Markers for an Acute Clinical Exacerbation in a CF Subject and/or a P. aeruginosa Infection

The present invention provides a method of diagnosis or prognosis of P. aeruginosa infection or an acute clinical exacerbation in a subject comprising detecting in a biological sample from said subject a protein of P. aeruginosa or mixtures thereof, wherein the presence of said protein in the sample is indicative of infection or exacerbation.

The present invention additionally provides a method of diagnosis or prognosis of P. aeruginosa infection or an acute clinical exacerbation in a subject comprising detecting in a biological sample from said subject an antibody to a protein of P. aeruginosa or mixtures thereof, wherein the presence of said protein in the sample is indicative of infection or exacerbation

As discussed supra, the term “a protein of P. aeruginosa” preferably encompasses a protein selected from the group consisting of HitA, PAPS, thioredoxin, GroES, NDK and DNA binding protein HU, or an immunogenic fragment or epitope thereof.

As used herein the term “Ferric iron-binding protein HitA” or “HitA” shall be taken to mean any peptide, polypeptide, or protein comprising an amino acid sequence at least about 80% identical to the amino acid sequence of a HitA set forth in SEQ ID NO: 1. The term “HitA” shall also be taken to include a peptide, polypeptide or protein having the known biochemical properties of HitA. As used herein the term “known biological properties” shall be understood to mean any physico-chemical properties by which a particular peptide, polypeptide, or protein may be characterised, such as, for example molecular weight, post-translational modifications, amino acid composition, or isoelectric point, amongst others.

Preferably, the percentage identity to SEQ ID NO: 1 is at least about 85%, more preferably at least about 90%, even more preferably at least about 95% and still more preferably at least about 99%.

In one embodiment, the HitA protein is a Pseudomonas protein.

In a particularly preferred embodiment, the HitA is P. aeruginosa HitA.

As used herein the term “thioredoxin dependent reductase” or “PAPS” shall be taken to mean any peptide, polypeptide, or protein comprising an amino acid sequence at least about 80% identical to the amino acid sequence of a PAPS set forth in SEQ ID NO: 2. The term “PAPS” shall also be taken to include a peptide, polypeptide or protein having the known biochemical properties of PAPS.

Preferably, the percentage identity to SEQ ID NO: 2 is at least about 85%, more preferably at least about 90%, even more preferably at least about 95% and still more preferably at least about 99%.

In one embodiment, the PAPS protein is a Pseudomonas PAPS protein.

In a particularly preferred embodiment, the PAPS is a P. aeruginosa PAPS.

As used herein the term “thioredoxin” shall be taken to mean any peptide, polypeptide, or protein comprising an amino acid sequence at least about 80% identical to the amino acid sequence of a thioredoxin forth in SEQ ID NO: 3 The term “thioredoxin” shall also be taken to include a peptide, polypeptide or protein having the known biochemical properties of thioredoxin.

Preferably, the percentage identity to SEQ ID NO: 3 is at least about 85%, more preferably at least about 90%, even more preferably at least about 95% and still more preferably at least about 99%.

In one embodiment, the thioredoxin protein is a Pseudomonas thioredoxin protein.

In a particularly preferred embodiment, the thioredoxin is P. aeruginosa thioredoxin.

As used herein the term an “heat shock protein GroES” or “GroES” shall be taken to mean any peptide, polypeptide, or protein comprising an amino acid sequence at least about 80% identical to the amino acid sequence set forth in SEQ ID NO: 4. The term “GroES” shall also be taken to include a peptide, polypeptide or protein having the known biochemical properties of GroES.

Preferably, the percentage identity to SEQ ID NO: 4 is at least about 85%, more preferably at least about 90%, even more preferably at least about 95% and still more preferably at least about 99%.

In one embodiment, the GroES protein is a Pseudomonas GroES protein.

In a particularly preferred embodiment, GroES is P. aeruginosa GroES.

As used herein the term an “nucleotide dependent kinase” or “NDK” shall be taken to mean any peptide, polypeptide, or protein comprising an amino acid sequence at least about 80% identical to the amino acid sequence set forth in SEQ ID NO: 5. The term “NDK” shall also be taken to include a peptide, polypeptide or protein having the known biochemical properties of NDK.

Preferably, the percentage identity to SEQ ID NO: 5 is at least about 85%, more preferably at least about 90%, even more preferably at least about 95% and still more preferably at least about 99%.

In one embodiment, the NDK protein is a Pseudomonas NDK protein.

In a particularly preferred embodiment, NDK is P. aeruginosa NDK.

As used herein the term an “DNA binding protein HU” shall be taken to mean any peptide, polypeptide, or protein comprising an amino acid sequence at least about 80% identical to the amino acid sequence set forth in SEQ ID NO: 6. The term “DNA binding protein HU” shall also be taken to include a peptide, polypeptide or protein having the known biochemical properties of DNA binding protein HU.

Preferably, the percentage identity to SEQ ID NO: 6 is at least about 85%, more preferably at least about 90%, even more preferably at least about 95% and still more preferably at least about 99%.

In one embodiment, the DNA binding protein HU proteins is a Pseudomonas DNA binding protein HU protein.

In a particularly preferred embodiment, DNA binding protein HU is P. aeruginosa DNA binding protein HU.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Maddison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximise the number of identical/similar residues and to minimise the number and/or length of sequence gaps in the alignment. Amino acid sequence alignments can also be performed using a variety of other commercially available sequence analysis programs, such as, for example, the BLAST program available at NCBI.

Preferred fragments if a protein of P. aeruginosa include those which include an epitope, in particular an epitope recognized by a B cell or a T cell.

An epitope recognized by a B-cell (i.e. a B-cell epitope) is conveniently derived from the amino acid sequence of an immunogenic protein of P. aeruginosa. Idiotypic and anti-idiotypic B cell epitopes against which an immune response is desired are specifically encompassed by the invention, as are lipid-modified B cell epitopes or a Group B protein. A preferred B-cell epitope is capable of eliciting the production of antibodies when administered to a mammal, preferably neutralizing antibody against P. aeruginosa, and more preferably, a high titer neutralizing antibody. Shorter B cell epitopes are preferred, to facilitate peptide synthesis. Preferably, the length of the B cell epitope will not exceed about 30 amino acids in length. More preferably, the B cell epitope sequence consists of about 25 amino acid residues or less, and more preferably less than 20 amino acid residues, and even more preferably about 5-20 amino acid residues in length derived from the sequence of the full-length protein of P. aeruginosa.

A CTL epitope is also conveniently derived from the full length amino acid sequence of a protein of P. aeruginosa and will generally consist of at least about 9 contiguous amino acids of said protein of P. aeruginosa and have an amino acid sequence that interacts at a significant level with a MHC Class I allele as determined using a predictive algorithm for determining MHC Class I-binding epitopes, such as, for example, the SYFPEITHI algorithm of the University of Tuebingen, Germany, or the algorithm of the HLA Peptide Binding Predictions program of the BioInformatics and Molecular Analysis Section (BIMAS) of the National Institutes of Health of the Government of the United States of America. More preferably, the CTL epitope will have an amino acid sequence that binds to and/or stabilizes a MHC Class I molecule on the surface of an antigen presenting cell (APC). Even more preferably, the CTL epitope will have a sequence that induces a memory CTL response or elicits IFN-γ expression by a T cell, such as, for example, CD8⁺ T cell, cytotoxic T cell (CTL). Still even more preferably, the CTL will have a sequence that stimulates CTL activity in a standard cytotoxicity assay. Preferred CTL epitopes of a protein of P. aeruginosa are capable of eliciting a cellular immune response against P. aeruginosa in human cells or tissues, such as, for example, by recognizing and lyzing human cells infected with P. aeruginosa, thereby providing or enhancing cellular immunity against P. aeruginosa.

Suitable fragments will be at least about 5, e.g. 10, 12, 15 or 20 amino acids in length. They may also be less than 200, 100 or 50 amino acids in length.

The amino acid sequence of a protein of P. aeruginosa or an immunogenic fragment or epitope thereof may be modified for particular purposes according to methods known to those of skill in the art without adversely affecting its immune function. For example, particular peptide residues are derivatized or chemically modified to enhance the immune response or to permit coupling of the peptide to other agents, e.g. lipids. It also is possible to change particular amino acids within the peptides without disturbing the overall structure or antigenicity of the peptide. Such changes are therefore termed “conservative” changes and tend to rely on the hydrophilicity or polarity of the residue. The size and/or charge of the side chains also are relevant factors in determining which substitutions are conservative.

Diagnostic/Prognostic Methods for Detecting P. aeruginosa Infection

### 1. Antigen-Based Assays

The diagnostic assays of the invention are useful for determining the progression of an infection by P. aeruginosa or an acute clinical exacerbation in a subject. In accordance with these diagnostic/prognostic applications of the invention, the level of protein of P. aeruginosa or an immunogenic fragment or epitope thereof in a biological sample is positively correlated with the infectious state. For example, a level of a protein of P. aeruginosa or an immunogenic fragment thereof that is less than the level of the same protein of P. aeruginosa or fragment detectable in a subject suffering from the symptoms of an exacerbation or an infection indicates that the subject is recovering from the infection. Similarly, a higher level of the protein or fragment in a sample from the subject compared to a healthy individual indicates that the subject has not been rendered free of the disease or infection.

Accordingly, a the present invention additionally provides a method for determining the response of a subject having an infection by P. aeruginosa or an acute clinical exacerbation to treatment with a therapeutic compound for said infection or exacerbation, said method comprising detecting a protein of P. aeruginosa or an immunogenic fragment or epitope thereof in a biological sample from said subject, wherein a level of the protein or fragment or epitope that is enhanced compared to the level of that protein or fragment or epitope detectable in a normal or healthy subject indicates that the subject is not responding to said treatment or has not been rendered free of exacerbation or infection.

In one embodiment, the method comprises contacting a biological sample derived from the subject with one or more antibodies or ligands capable of binding to a protein of P. aeruginosa or an immunogenic fragment or epitope thereof, and detecting the formation of an antigen-antibody/ligand complex.

In an alternative embodiment, the present invention provides a method for determining the response of a subject having an infection by P. aeruginosa or an acute clinical exacerbation to treatment with a therapeutic compound for said infection or exacerbation, said method comprising detecting a protein of P. aeruginosa or an immunogenic fragment or epitope thereof in a biological sample from said subject, wherein a level of the protein or fragment or epitope that is lower than the level of the protein or fragment or epitope detectable in a subject suffering from infection by P. aeruginosa or exacerbation indicates that the subject is responding to said treatment or has been rendered free of disease or infection. Clearly, if the level of the protein of P. aeruginosa or fragment or epitope thereof is not detectable in the subject, the subject has responded to treatment.

In one embodiment, the method comprises method comprises contacting a biological sample derived from the subject with one or more antibodies or ligands capable of binding to a protein of P. aeruginosa or an immunogenic fragment or epitope thereof, and detecting the formation of an antigen-antibody complex.

In a further embodiment, the amount of a protein of P. aeruginosa in a biological sample derived from a patient is compared to the amount of the same protein detected in a biological sample previously derived from the same patient. As will be apparent to a person skilled in the art, this method may be used to continually monitor a patient with an infection or exacerbation. In this way a patient may be monitored for the onset or progression of an infection or exacerbation.

Alternatively, or in addition, the amount of a protein detected in a biological sample derived from a subject with an infection or exacerbation may be compared to a reference sample, wherein the reference sample is derived from one or more subjects that do not suffer from an infection or exacerbation or alternatively, one or more subjects that have recently received successful treatment for infection and/or one or more subjects that do not suffer from an infection or exacerbation.

In one embodiment, a protein of P. aeruginosa or immunogenic fragment thereof is not detected in a reference sample, however, said protein of P. aeruginosa or immunogenic fragment thereof is detected in the patient sample, indicating that the patient from whom the sample was derived is suffering from infection by P. aeruginosa or an exacerbation or will develop an acute infection or exacerbation.

Alternatively, the amount of the protein of P. aeruginosa or immunogenic fragment thereof may be enhanced in the patient sample compared to the level detected in a reference sample. Again, this indicates that the patient from whom the biological sample was isolated is suffering from infection by P. aeruginosa or an exacerbation or will develop an acute infection or exacerbation.

In one embodiment of the diagnostic/prognostic methods described herein, the biological sample is obtained previously from the subject. In accordance with such an embodiment, the prognostic or diagnostic method is performed ex vivo.

In yet another embodiment, the subject diagnostic/prognostic methods further comprise processing the sample from the subject to produce a derivative or extract that comprises the analyte (e.g., pleural fluid or sputum).

Suitable samples include extracts from tissues such as brain, breast, ovary, lung, colon, pancreas, testes, liver, muscle, skin or a sample from skin (e.g. a skin swab) and bone tissues, or body fluids such as sputum, serum, plasma, whole blood, sera, urine, saliva or pleural fluid.

Preferably, the biological sample is a bodily fluid or tissue sample or is derived from a body fluid or tissue sample selected from the group consisting of: blood, serum, sputum, saliva, urine, and lung. Other samples are not excluded.

In a preferred embodiment, the present invention provides a method for diagnosing an infection by P. aeruginosa in a subject comprising contacting a biological sample derived from the subject with one or more antibodies or ligands capable of binding to ferric iron-binding protein (HitA) or an immunogenic fragment or epitope thereof for a time and under conditions sufficient for an antibody/ligand-antigen complex to form, and detecting the antigen-antibody complex.

In another embodiment, the present invention provides a method for diagnosing an infection by P. aeruginosa in a subject comprising contacting a biological sample derived from the subject with one or more antibodies or ligands capable of binding to thioredoxin dependent reductase (PAPS) or an immunogenic fragment or epitope thereof for a time and under conditions sufficient for an antibody/ligand-antigen complex to form, and detecting the antigen-antibody complex.

In a further embodiment, the present invention provides a method for diagnosing an infection by P. aeruginosa in a subject comprising contacting a biological sample derived from the subject with one or more antibodies or ligands capable of binding to thioredoxin or an immunogenic fragment or epitope thereof for a time and under conditions sufficient for an antibody/ligand-antigen complex to form, and detecting the antigen-antibody complex.

In a still further preferred embodiment, the present invention provides a method for diagnosing an infection by P. aeruginosa in a subject comprising contacting a biological sample derived from the subject with one or more antibodies capable of binding to heat shock protein GroES or an immunogenic fragment or epitope thereof for a time and under conditions sufficient for an antibody/ligand-antigen complex to form, and detecting the antigen-antibody complex.

In another preferred embodiment, the present invention provides a method for diagnosing an infection by P. aeruginosa in a subject comprising contacting a biological sample derived from the subject with one or more antibodies or ligands capable of binding to nucleotide dependent kinase (NDK) or an immunogenic fragment or epitope thereof for a time and under conditions sufficient for an antibody/ligand-antigen complex to form, and detecting the antigen-antibody complex.

The present invention additionally provides a method for diagnosing an infection by P. aeruginosa in a subject comprising contacting a biological sample derived from the subject with one or more antibodies or ligands capable of binding to DNA-binding protein HU or an immunogenic fragment or epitope thereof for a time and under conditions sufficient for an antibody/ligand-antigen complex to form, and detecting the antigen-antibody complex.

### 2. Antibody-Based Assays

The present invention also provides a method of diagnosing an infection by P. aeruginosa or an acute clinical exacerbation in a subject comprising detecting in a biological sample from said subject antibodies against a protein of P. aeruginosa or an immunogenic fragment or epitope thereof, wherein the presence of said antibodies in the sample is indicative of infection or exacerbation. The infection may be a past or present infection, or a latent infection.

In one embodiment, the method comprises contacting the biological sample with a peptide mimetic of a protein of P. aeruginosa or a fragment or epitope thereof.

Alternatively, the present invention provides a method for detecting P. aeruginosa infection in a subject, the method comprising contacting a biological sample derived from the subject with a protein of P. aeruginosa or an immunogenic fragment or epitope thereof and detecting the formation of an antigen-antibody complex wherein detection of the complex is indicative of infection.

In another embodiment, the diagnostic assays of the invention are useful for determining the progression of an infection by P. aeruginosa or an acute clinical exacerbation in a subject. In accordance with these prognostic applications of the invention, the amount of antibodies against a protein of P. aeruginosa or fragment or epitope in blood or serum or urine or an immunoglobulin fraction from the subject is positively correlated with the infectious state or state of exacerbation. For example, a level of the antibodies that is less than the level of the antibodies detectable in a subject suffering from the P. aeruginosa infection indicates that the subject is recovering from the infection. Similarly, a higher level of the antibodies in a sample from the subject compared to a healthy individual indicates that the subject has not been rendered free of the exacerbation or infection.

In a further embodiment, the present invention provides a method for determining the response of a subject having a P. aeruginosa infection or suffering from an acute clinical exacerbation treatment with a therapeutic compound for said infection or exacerbation, said method comprising detecting antibodies against a protein of P. aeruginosa or an immunogenic fragment or epitope thereof in a biological sample from said subject, wherein a level of the antibodies that is enhanced compared to the level of the antibodies detectable in a normal or healthy subject indicates that the subject is not responding to said treatment or has not been rendered free of disease or infection.

In an alternative embodiment, the present invention provides a method for determining the response of a subject having an infection by P. aeruginosa or suffering from an acute clinical exacerbation to treatment with a therapeutic compound for said infection or exacerbation, said method comprising detecting antibodies against a protein of P. aeruginosa or an immunogenic fragment or epitope thereof in a biological sample from said subject, wherein a level of the antibodies that is lower than the level of the antibodies detectable in a subject suffering from infection by P. aeruginosa or an exacerbation indicates that the subject is responding to said treatment or has been rendered free of disease or infection.

In one embodiment of the diagnostic/prognostic methods described herein, the biological sample is obtained previously from the subject. In accordance with such an embodiment, the prognostic or diagnostic method is performed ex vivo.

In yet another embodiment, the subject diagnostic/prognostic methods further comprise processing the sample from the subject to produce a derivative or extract that comprises the analyte (blood, serum, urine or immunoglobulin-containing fraction).

In one embodiment, the method of the invention provides, a method for diagnosing an infection by P. aeruginosa in a subject comprising contacting a biological sample derived from the subject with ferric iron-binding protein (HitA) or an immunogenic fragment or epitope thereof for a time and under conditions sufficient for an antibody-antigen complex to form, and detecting the antigen-antibody complex.

In another embodiment, the present invention provides a method for diagnosing an infection by P. aeruginosa in a subject comprising contacting a biological sample derived from the subject with a thioredoxin dependent reductase (PAPS) or an immunogenic fragment or epitope thereof for a time and under conditions sufficient for an antibody-antigen complex to form and detecting the antigen-antibody complex.

In a further embodiment, the present invention provides a method for diagnosing an infection by P. aeruginosa in a subject comprising contacting a biological sample derived from the subject with a thioredoxin or an immunogenic fragment or epitope thereof for a time and under conditions sufficient for and antibody-antigen complex to form and detecting the antigen-antibody complex.

In a still further embodiment, the present invention provides a method for diagnosing an infection by P. aeruginosa in a subject comprising contacting a biological sample derived from the subject with a heat shock protein GroES or an immunogenic fragment or epitope thereof for a time and under conditions sufficient for an antibody-antigen complex to form and detecting the antigen-antibody complex.

In yet another embodiment, the present invention provides a method for diagnosing an infection by P. aeruginosa in a subject comprising contacting a biological sample derived from the subject with a nucleotide dependent kinase (NDK) or an immunogenic fragment or epitope thereof for a time and under conditions sufficient for an antibody-antigen complex to form and detecting the antigen-antibody complex.

In a still further embodiment, the present invention provides a method for diagnosing an infection by P. aeruginosa in a subject comprising contacting a biological sample derived from the subject with a DNA-binding protein HU or an immunogenic fragment or epitope thereof, for a time and under conditions sufficient for an antibody-antigen complex to form and detecting the antigen-antibody complex.

The present invention clearly contemplates a multiplex assay. For example, the present invention clearly contemplates the detection of a plurality of proteins of P. aeruginosa or a fragment or epitope thereof in a sample from a subject. Alternatively, or in addition the present invention contemplates the detection of a plurality of antibodies against a plurality of proteins of P. aeruginosa or a fragment or epitope thereof in a sample from a subject. Clearly, the present invention additionally contemplates the detection of one or more proteins of P. aeruginosa or a fragment or epitope thereof in a sample from a subject and antibodies against one or more proteins of P. aeruginosa or a fragment or epitope thereof in a sample from the subject.

### 3. Detection Systems

Preferred detection systems contemplated herein include any known assay for detecting proteins or antibodies in a biological sample isolated from a human subject, such as, for example, SDS/PAGE, isoelectric focussing, 2-dimensional gel electrophoresis comprising SDS/PAGE and isoelectric focussing, an immunoassay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these embodiments, the antibody or ligand may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, particularly a high throughput spectroscopy resonance method or immunoassay are contemplated.

Immunoassay formats are preferred, e.g., selected from the group consisting of, an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), biosensor technology, evanescent fiber-optics technology or protein chip technology are also useful.

Preferably, the assay is a semi-quantitative assay or quantitative assay.

Standard solid phase ELISA formats are useful in determining the concentration of a protein or antibody from a variety of patient samples.

In one form such as an assay involves immobilising a biological sample comprising antibodies to a protein of P. aeruginosa, or alternatively, a protein of P. aeruginosa or an immunogenic fragment thereof, onto a solid matrix, such as, for example a polystyrene or polycarbonate microwell or dipstick, a membrane, or a glass support (e.g. a glass slide).

In the case of an antigen-based assay, an antibody that specifically binds a protein of P. aeruginosa or fragment thereof is brought into direct contact with the immobilised biological sample, and forms a direct bond with any of its target protein present in said sample. For an antibody-based assay, an immobilised isolated or recombinant protein of P. aeruginosa or an immunogenic fragment or epitope thereof is contacted with the biological sample. The added antibody or protein in solution is generally labelled with a detectable reporter molecule, such as for example, a fluorescent label (e.g. FITC or Texas Red) or an enzyme (e.g. horseradish peroxidase (HRP)), alkaline phosphatase (AP) or β-galactosidase. Alternatively, or in addition, a second labelled antibody can be used that binds to the first antibody or to the isolated/recombinant protein of P. aeruginosa or fragment thereof. Following washing to remove any unbound antibody or antigen, the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal).

Such ELISA based systems are suitable for quantification of the amount of a protein or antibody in a sample, such as, for example, by calibrating the detection system against known amounts of a standard.

In another form, an ELISA consists of immobilizing an antibody that specifically binds a protein of P. aeruginosa on a solid matrix, such as, for example, a membrane, a polystyrene or polycarbonate microwell, a polystyrene or polycarbonate dipstick or a glass support. A patient sample is then brought into physical relation with said antibody, and the antigen in the sample is bound or ‘captured’. The bound protein is then detected using a labelled antibody. Alternatively, a third labelled antibody can be used that binds the second (detecting) antibody.

It will be apparent to the skilled person that the assay formats described herein are amenable to high throughput formats, such as, for example automation of screening processes, or a microarray format as described in Mendoza et al, Biotechniques 27(4): 778-788, 1999. Furthermore, variations of the above described assay will be apparent to those skilled in the art, such as, for example, a competitive ELISA.

Alternatively, the presence of antibodies to a protein associated with anaerobic growth of P. aeruginosa, or alternatively a protein of P. aeruginosa or an immunogenic fragment thereof, is detected using a radioimmunoassay (RIA). The basic principle of the assay is the use of a radiolabelled antibody or antigen to detect antibody antigen interactions. For example, an antibody that specifically binds to a protein of P. aeruginosa is bound to a solid support and a biological sample brought into direct contact with said antibody. To detect the bound antigen, an isolated and/or recombinant form of the antigen is radiolabelled is brought into contact with the same antibody. Following washing the amount of bound radioactivity is detected. As any antigen in the biological sample inhibits binding of the radiolabelled antigen the amount of radioactivity detected is inversely proportional to the amount of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

Western blotting is also useful for detecting a protein of P. aeruginosa or an immunogenic fragment thereof. In such an assay protein from a biological sample is separated using sodium dodecyl sulphate (SDS) polyacrylamide gel electrophoresis (SDS-PAGE) or native gel electrophoresis using techniques known in the art and/or described in, for example, Scopes (In: Protein Purification: Principles and Practice, Third Edition, Springer Verlag, 1994). Separated proteins are then transferred to a solid support, such as, for example, a membrane or more specifically PVDF membrane, using a method known in the art, for example, electrotransfer. This membrane is then blocked and probed with a labelled antibody or ligand that specifically binds a protein of P. aeruginosa. Alternatively, a labelled secondary, or even tertiary, antibody or ligand can be used to detect the binding of a specific primary antibody.

High-throughput methods for detecting the presence or absence of antibodies that bind to a protein of P. aeruginosa, or alternatively a protein of P. aeruginosa or an immunogenic fragment thereof are particularly preferred.

For example, a biosensor is used. Biosensor devices generally employ an electrode surface in combination with current or impedance measuring elements to be integrated into a device in combination with the assay substrate (such as that described in U.S. Pat. No. 5,567,301). An antibody or ligand that specifically binds to a protein of interest is preferably incorporated onto the surface of a biosensor device and a biological sample isolated from a patient (for example sputum that has been or serum) contacted to said device. A change in the detected current or impedance by the biosensor device indicates protein binding to said antibody or ligand. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of use in high throughput analysis due to the ease of adapting such systems to micro- or nano-scales. Furthermore, such systems are conveniently adapted to incorporate several detection reagents, allowing for multiplexing of diagnostic reagents in a single biosensor unit for example, to detect the level of a number of proteins associated with anaerobic growth of P. aeruginosa or antibodies that bind thereto. This permits the simultaneous detection of several epitopes in a small amount of body fluids.

Evanescent biosensors are also preferred as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the diagnostic protein to the antibody or ligand.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods for generating a protein chip are known in the art and are described in for example U.S. Patent Application No. 20020136821, 20020192654, 20020102617 and U.S. Pat. No. 6,391,625. To bind a protein to a solid support it is often necessary to treat the solid support so as to create chemically reactive groups on the surface, such as, for example, with an aldehyde-containing silane reagent. Alternatively, an antibody or ligand is captured on a microfabricated polyacrylamide gel pad and accelerated into the gel using microelectrophoresis as described in, Arenkov et al. Anal. Biochem. 278:123-131, 2000. Alternatively, a protein or an antibody that binds thereto is “spotted” onto a solid support, e.g. a membrane, using a method known in the art and/or exemplified herein.

A protein chip is preferably generated such that several proteins, ligands or antibodies are arrayed on said chip. This format permits the simultaneous screening for the presence of several proteins in a sample (e.g. several proteins associated with anaerobic growth of P. aeruginosa or antibodies that bind thereto).

Alternatively, a protein chip may comprise only one protein, ligand or antibody, and be used to screen one or more patient samples for the presence of the polypeptide or antibody of interest. Such a chip may also be used to simultaneously screen an array of patient samples for a polypeptide or antibody of interest.

A sample to be analysed using a protein chip may be attached to a reporter molecule, such as, for example, a fluorescent molecule, a radioactive molecule, an enzyme, or an antibody that is detectable using methods known in the art. Accordingly, by contacting a protein chip with a labelled sample and subsequent washing to remove any unbound proteins the presence of a bound protein is detected using methods well known in the art, such as, for example using a DNA microarray reader.

Alternatively, the binding of a polypeptide or antibody is detected with a labelled antibody or ligand.

Alternatively, biomolecular interaction analysis-mass spectrometry (BIA-MS) is used to rapidly detect and characterise a protein present in complex biological samples at the low- to sub-fmole level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterise a protein bound to the protein chip. Alternatively, the protein chip is analysed using ESI as described in U.S. Patent Application 20020139751.

As will be apparent to the skilled artisan, protein chips are amenable to multiplexing of detection reagents. Accordingly, several antibodies or ligands each able to specifically bind a different peptide or protein may be bound to different regions of said protein chip. Analysis of a biological sample using said chip then permits the detecting of multiple proteins of interest, or multiple B cell epitopes, e.g., of a protein of P. aeruginosa, (e.g., a protein selected from the group consisting of HitA, PAPS, thioredoxin, GroES, NDK and DNA binding protein HU or mixtures thereof). Multiplexing of diagnostic and prognostic markers is contemplated in the present invention.

As will be apparent from the preceding discussion, it is preferable that a detection system that is antibody or ligand based is used in the method of the present invention. Immunoassay formats are even more preferred.

Clearly, any antibody or ligand for use (or when used) in such an assay is encompassed by the instant invention. Methods for the production of such an antibody or ligand are known in the art and described herein.

### Antibodies

As used herein the term “antibody” refers to intact monoclonal or polyclonal antibodies, immunoglobulin (IgA, IgD, IgG, IgM and/or IgE) fractions, humanized antibodies, or recombinant single chain antibodies, as well as fragments thereof, such as, for example Fab, F(ab)2, and Fv fragments.

Antibodies referred to herein are obtained from a commercial source, or alternatively, produced by conventional means. Commercial sources are well known to those skilled in the art.

High titer antibodies are preferred, as these are more useful commercially in kits for diagnostic or therapeutic applications. By “high titer” is meant a titer of at least about 1:10³ or 1:10⁴ or 1:10⁵. Methods of determining the titer of an antibody will be apparent to the skilled artisan. For example, the titer of an antibody in purified antiserum may be determined using an ELISA assay to determine the amount of IgG in a sample. Typically an anti-IgG antibody or Protein G is used to bind the IgG. The amount detected in a sample is compared to a control sample of a known amount of purified and/or recombinant IgG to determine the actual amount of IgG. Alternatively, a kit for determining antibody may be used, e.g. the Easy TITER kit from Pierce (Rockford, Ill., USA).

Antibodies are preferably prepared by any of a variety of techniques known to those of ordinary skill in the art, and described, for example in, Harlow and Lane (In: Antibodies: A Laboratory Manual, Cold Spring Harbor Laboratory, 1988). In one such technique, an immunogen comprising the antigenic polypeptide (e.g. a protein of P. aeruginosa or an immunogenic fragment thereof) is initially injected into any of a wide variety of mammals (e.g., a mouse, a rat, a rabbit, a sheep, a dog, a pig, a chicken or a goat). The immunogen is derived from a natural source, produced by recombinant expression means, or artificially generated, such as by chemical synthesis (e.g., BOC chemistry or FMOC chemistry). In this step, the polypeptides or fragments thereof of this invention may serve as the immunogen without modification. Alternatively, a peptide, polypeptide or protein is joined to a carrier protein, such as bovine serum albumin or keyhole limpet hemocyanin. The immunogen and optionally a carrier for the protein is injected into the animal host, preferably, according to a predetermined schedule incorporating one or more booster immunizations, and blood collected from said the animals periodically. Optionally the immunogen is injected in the presence of an adjuvant, such as, for example Freund's complete or incomplete adjuvant, lysolecithin or dinitrophenol to enhance the immune response to the immunogen. Monoclonal or polyclonal antibodies specific for the polypeptide may then be purified from the blood isolated form an animal by, for example, affinity chromatography using the polypeptide coupled to a suitable solid support.

Monoclonal antibodies specific for the antigenic polypeptide of interest are prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest) e.g. a protein of P. aeruginosa or an immunogenic fragment thereof. Such cell lines are produced, for example, from spleen cells obtained from an animal immunized as described above. The spleen cells are then immortalized by, for example, fusion with a myeloma cell fusion partner, preferably one that is syngenic with the immunized animal. Any of a variety of fusion techniques is employed, for example, the spleen cells and myeloma cells are combined with a nonionic detergent or electrofused and then grown in a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and growth media in which the cells have been grown is tested for the presence of binding activity against the polypeptide (immunogen) of interest. Hybridomas having high reactivity and specificity for the polypeptide (immunogen) of interest are preferred.

Monoclonal antibodies are isolated from the supernatants of growing hybridoma colonies using methods such as, for example, affinity purification. In addition, various techniques are employed to enhance the yield, such as injection of the hybridoma cell line into the peritoneal cavity of a suitable vertebrate host, such as a mouse. Monoclonal antibodies are then harvested from the ascites fluid or the blood. Contaminants are removed from the antibodies by conventional techniques, such as chromatography, gel filtration, precipitation, and extraction. The polypeptides of this invention may be used in the purification process in, for example, an affinity chromatography step.

Alternatively, ABL-MYC technology (NeoClone, Madison Wis. 53713, USA) is used to produce cell lines secreting monoclonal antibodies (mAbs) against a protein of P. aeruginosa or a fragment or epitope thereof. In this process, BALB/cByJ female mice are immunized with an amount of the peptide antigen over a period of about 2 to about 3 months. During this time, test bleeds are taken from the immunized mice at regular intervals to assess antibody responses in a standard ELISA. The spleens of mice having antibody titers of at least about 1,000 are used for subsequent ABL-MYC infection employing replicaton-incompetent retrovirus comprising the oncogenes v-abl and c-myc. Splenocytes are transplanted into naive mice which then develop ascites fluid containing cell lines producing monoclonal antibodies (mAbs) against the protein of P. aeruginosa or a fragment or epitope thereof. The mAbs are purified from ascites using protein G or protein A, e.g., bound to a solid matrix, depending on the isotype of the mAb. Because there is no hybridoma fusion, an advantage of the ABL-MYC process is that it is faster, more cost effective, and higher yielding than conventional mAb production methods. In addition, the diploid palsmacytomas produced by this method are intrinsically more stable than polyploid hybridomas, because the ABL-MYC retrovirus infects only cells in the spleen that have been stimulated by the immunizing antigen. ABL-MYC then transforms those activated B-cells into immortal, mAb-producing plasma cells called plasmacytomas. A “plasmacytoma” is an immortalized plasma cell that is capable of uncontrolled cell division. Since a plasmacytoma begins with just one cell, all of the plasmacytomas produced from it are therefore identical, and moreover, produce the same desired “monoclonal” antibody. As a result, no sorting of undesirable cell lines is required. The ABL-MYC technology is described generically in detail in the following disclosures which are incorporated by reference herein:
1. Largaespada et al., Curr. Top. Microbiol. Immunol., 166, 91-96. 1990;
2. Weissinger et al., Proc. Natl. Acad. Sci. USA, 88, 8735-8739, 1991;
3. Largaespada et al., Oncogene, 7, 811-819, 1992;
4. Weissinger et al., J. Immunol. Methods 168, 123-130, 1994;
5. Largaespada et al., J. Immunol. Methods. 197(1-2), 85-95, 1996; and
6. Kumar et al., Immuno. Letters 65, 153-159, 1999.

As discussed supra antibody fragments are contemplated by the present invention. The term “antibody fragment” refers to a portion of a full-length antibody, generally the antigen binding or variable region. Examples of antibody fragments include Fab, Fab′, F(ab′)₂ and Fv fragments.

Papain digestion of an antibody produces two identical antigen binding fragments, called the Fab fragment, each with a single antigen binding site, and a residual “Fc” fragment.

Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

An “Fv” fragment is the minimum antibody fragment that contains a complete antigen recognition and binding site. This region consists of a dimer of one heavy and one light chain variable domain in a non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen.

A Fab fragment [also designated as F(ab)] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

“Single-chain Fv” or “scFv” antibody fragments comprise the VH and VL domains of an antibody, wherein these domains are present in a single polypeptide chain. Generally, the Fv polypeptide further comprises a polypeptide linker between the VH and VL domains which enables the scFv to form the desired structure for antigen binding. For a review of scFv, see Pluckthun in The Pharmacology of Monoclonal Antibodies, vol. 113, Rosenburg and Moore eds. Springer-Verlag, New York, pp. 269-315 (1994).

As will be apparent from the preceding paragraph, an antibody useful for the method of the invention is a recombinant antibody, such as, for example, a recombinant ScFv antibody fragment. Essentially, a ScFv antibody fragment is a recombinant single chain molecule containing the variable region of a light chain of an antibody and the variable region of a heavy chain of an antibody, linked by a suitable, flexible polypeptide linker.

A library of ScFv fragments is produced, for example, by amplifying the variable regions of a large and/or small chain from nucleic acid encoding an immunoglobulin (for example, using nucleic acid from a spleen cell that may or may not be derived from a subject (e.g., a mouse) that has been previously immunized a protein of P. aeruginosa or a fragment or epitope thereof). These regions are cloned into a vector encoding a suitable framework including a linker region to facilitate expression, for example, on the surface of a phage or in a cell, in the case of an intrabody (for example, see Worn et al., J. Biol. Chem., 275: 2795-803, 2003). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., Mol. Cell. Biol. 15:1182-91, 1995; Lener et al., Eur. J. Biochem. 267:1196-205 2000).

Alternatively, a library of recombinant antibodies is screened using phage display or in vitro display, for example, as described in Rauchenberger et al., J. Biol. Chem. 278:38194-205, 2003.

It is preferable that an immunogen used in the production of an antibody is one that is sufficiently antigenic to stimulate the production of antibodies that will bind to the immunogen and is preferably, a high titer antibody. In one embodiment, an immunogen is an entire protein.

In another embodiment, an immunogen consists of a peptide or a fragment of a peptide derived from a protein associated with anaerobic growth of P. aeruginosa. Preferably, an antibody raised to such an immunogen also recognises the full-length protein from which the immunogen was derived, such as, for example, in its native state or having a native conformation.

Alternatively, or in addition, an antibody raised against a peptide immunogen recognises the full-length protein from which the immunogen was derived when the protein is denatured. By “denatured” is meant that conformational epitopes of the protein are disrupted under conditions that retain linear B cell epitopes of the protein. As will be known to a skilled artisan linear epitopes and conformational epitopes may overlap.

In one embodiment, a peptide immunogen is determined using the method described by Hopp, Peptide Research, 6: 183-190 1993, wherein a hydrophilic peptide is selected as it is more likely to occur at the surface of the native protein. However, a peptide should not be too highly charged, as this may reduce the efficiency of antibody generation.

In another embodiment, a peptide immunogen is determined using the method described by Palfreyman et al J. Immunol. Meth. 75, 383-393, 1984, wherein the amino- and/or carboxy-terminal amino acids are used to generate a peptide against which specific antibodies are raised.

In yet another embodiment, a peptide immunogen is predicted using an algorithm such as for example that described in Kolaskar and Tongaonkar FEBS Lett. 2 76(1-2) 172-174, 1990. Such methods are based upon determining the hydrophilicity of regions of a protein, usually 6 amino acids, and determining those hydrophilic regions that are associated with turns in proteins, surface flexibility, or secondary structures, and are unlikely to be modified at the post-translational level, such as, for example by glycosylation. Such regions of a protein are therefore likely to be exposed, that is, at the surface of the three-dimensional structure of the protein. Furthermore, as these regions are not modified, they are likely to remain constant and as such offer a likely site of antibody recognition.

In yet another embodiment, overlapping peptides spanning the entire protein of interest, or a region of said protein are generated by synthetic means, using techniques well known in the art. Alternatively, a relatively short protein of low abundance or a portion of a protein that is difficult to purify from a natural source, is produced chemically (e.g. by BOC chemistry or FMOC chemistry).

Synthetic peptides are prepared using known techniques of solid phase, liquid phase, or peptide condensation, or any combination thereof, and can include natural and/or unnatural amino acids. Amino acids used for peptide synthesis may be standard Boc (Na-amino protected Na-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Na-amino protected 9-fluorenylmethoxycarbonyl (Fmoc) amino acids described by Carpino and Han, J. Org. Chem., 37:3403-3409, 1972. Both Fmoc and Boc Na-amino protected amino acids are obtainable from various commercial sources, such as, for example, Fluka, Bachem, Advanced Chemtech, Sigma, Cambridge Research Biochemical, Bachem, or Peninsula Labs.

Synthetic peptides are then optionally screened to determine linear B cell epitopes, using techniques known in the art. In one embodiment, the peptides are screened using an ELISA based assay to determine those peptides against which a subject with a P. aeruginosa infection has raised specific antibodies. Preferred peptides are those against which a subject with a P. aeruginosa infection has raised specific antibodies, but a subject not suffering said infection, or a healthy individual has not. Any peptide identified in such a screen is of use in a peptide based diagnostic or prognostic test.

Alternatively, or in addition, such an immunogenic peptide is used to generate a monoclonal or polyclonal antibody using methods known in the art, such as, for example, those described herein. The antibody is then tested to determine its specificity and sensitivity using, for example, an ELISA based assay.

As will be apparent to those skilled in the art a diagnostic or prognostic assay described herein may be a multiplexed assay. As used herein the term “multiplex”, shall be understood not only to mean the detection of two or more diagnostic or prognostic markers in a single sample simultaneously, but also to encompass consecutive detection of two or more diagnostic or prognostic markers in a single sample, simultaneous detection of two or more diagnostic or prognostic markers in distinct but matched samples, and consecutive detection of two or more diagnostic or prognostic markers in distinct but matched samples. As used herein the term “matched samples” shall be understood to mean two or more samples derived from the same initial biological sample, or two or more biological samples isolated at approximately the same point in time.

Accordingly, a multiplexed assay may comprise an assay that detects several antibodies that bind to and/or a protein of P. aeruginosa in the same reaction and simultaneously, or alternatively, it detects other one or more antigens/antibodies in addition to one or more antibodies that bind to and/or a protein of P. aeruginosa.

### Ligands

As used herein the term “ligand” shall be taken in its broadest context to include any chemical compound, polynucleotide, peptide, protein, lipid, carbohydrate, small molecule, natural product, polymer, etc. that is capable of selectively binding, whether covalently or not, to one or more proteins of P. aeruginosa or a fragment or an epitope thereof. The ligand may bind to its target via any means including hydrophobic interactions, hydrogen bonding, electrostatic interactions, van der Waals interactions, pi stacking, covalent bonding, or magnetic interactions amongst others.

In a preferred embodiment of the invention, the ligand is a peptidyl ligand.

Such a peptidyl compound may be produced using any means known in the art. For example, a peptidyl compound is produced synthetically. Synthetic peptides are prepared using known techniques of solid phase, liquid phase, or peptide condensation, or any combination thereof, and can include natural and/or unnatural amino acids. Amino acids used for peptide synthesis may be standard Boc (Na-amino protected Na-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Na-amino protected 9-fluorenylmethoxycarbonyl (Fmoc) amino acids described by Carpino and Han, J. Org. Chem., 37:3403-3409, 1972. Both Fmoc and Boc Na-amino protected amino acids can be obtained from various commercial sources, such as, for example, Fluka, Bachem, Advanced Chemtech, Sigma, Cambridge Research Biochemical, Bachem, or Peninsula Labs.

Alternatively, a synthetic peptide is produced using a technique known in the art and described, for example, in Stewart and Young (In: Solid Phase Synthesis, Second Edition, Pierce Chemical Co., Rockford, Ill. (1984) and/or Fields and Noble (Int. J. Pept. Protein Res., 35:161-214, 1990), or using an automated synthesizer. Accordingly, peptides of the invention may comprise D-amino acids, a combination of D- and L-amino acids, and various unnatural amino acids (e.g., β-methyl amino acids, Cα-methyl amino acids, and Nα-methyl amino acids, etc) to convey special properties. Synthetic amino acids include ornithine for lysine, fluorophenylalanine for phenylalanine, and norleucine for leucine or isoleucine.

In another embodiment, a peptidyl agent is produced using recombinant means. For example, an oligonucleotide or other nucleic acid is placed in operable connection with a promoter. Methods for producing such expression constructs, introducing an expression construct into a cell and expressing and/or purifying the expressed peptide, polypeptide or protein are known in the art and/or described herein.

Alternatively, the peptide, polypeptide or protein is expressed using a cell free system, such as, for example, the TNT system available from Promega. Such an in vitro translation system is useful for screening a peptide library by, for example, ribosome display, covalent display or mRNA display.

In a preferred embodiment, a peptide library is screened to identify a compound that binds to a protein of P. aeruginosa or an epitope or a fragment thereof. Suitable methods for production of such a library will be apparent to the skilled artisan and/or described herein.

For example, a random peptide library is produced by synthesizing random oligonucleotides of sufficient length to encode a peptide of desired length, e.g., 7 or 9 or 15 amino acids. Methods for the production of an oligonucleotide are known in the art. For example, an oligonucleotide is produced using standard solid-phase phosphoramidite chemistry. Essentially, this method uses protected nucleoside phosphoramidites to produce a short oligonucleotide (i.e., up to about 80 nucleotides). Typically, an initial 5′-protected nucleoside is attached to a polymer resin by its 3′-hydroxy group. The 5′ hydroxyl group is then de-protected and the subsequent nucleoside-3′-phosphoramidite in the sequence is then coupled to the de-protected group. The internucleotide bond is then formed by oxidising the linked nucleosides to form a phosphotriester. By repeating the steps of de-protection, coupling and oxidation an oligonucleotide of desired length and sequence is obtained. Suitable methods of oligonucleotide synthesis are described, for example, in Caruthers, M. H., et al., “Methods in Enzymology,” Vol. 154, pp. 287-314 (1988).

Each of the oligonucleotides is then inserted into an expression construct (in operable connection with a promoter) and introduced into a cell of the invention. Suitable methods for producing a random peptide library are described, for example, in Oldenburg et al., Proc. Natl. Acad. Sci. USA 89:5393-5397, 1992; Valadon et al., J. Mol. Biol., 261:11-22, 1996; Westerink Proc. Natl. Acad. Sci. USA., 92:4021-4025, 1995; or Felici, J. Mol. Biol., 222:301-310, 1991.

Optionally, the nucleic acid is positioned so as to produce a fusion protein, wherein the random peptide is conformationally constrained within a scaffold structure, e.g., a thioredoxin (Trx) loop (Blum et al. Proc. Natl. Acad. Sci. USA, 97, 2241-2246, 2000) or a catalytically inactive staphylococcal nuclease (Norman et al, Science, 285, 591-595, 1999), to enhance their stability. Such conformational constraint within a structure has been shown, in some cases, to enhance the affinity of an interaction between a random peptide and its target, presumably by limiting the degrees of conformational freedom of the peptide, and thereby minimizing the entropic cost of binding.

Alternatively, a ligand is a nucleic acid. For example, a nucleic acid aptamer (adaptable oligomer) is a nucleic acid molecule that is capable of forming a secondary and/or tertiary structure that provides the ability to bind to a molecular target. For example, an aptamer is produced that is capable of binding to a protein of P. aeruginosa or a fragment or epitope thereof. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990.

In another embodiment, the ligand is a small molecule. Techniques for synthesizing small organic compounds will vary considerably depending upon the compound, however such methods will be well known to those skilled in the art. In one embodiment, informatics is used to select suitable chemical building blocks from known compounds, for producing a combinatorial library. For example, QSAR (Quantitative Structure Activity Relationship) modelling approach uses linear regressions or regression trees of compound structures to determine suitability. The software of the Chemical Computing Group, Inc. (Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and logP (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, information from known appetite suppressants and non-suppressants, including lead compounds identified in initial screens, can be used to expand the list of compounds being screened to thereby identify highly active compounds.

### 4. Biological Samples and Reference Samples

Suitable biological samples are preferably isolated from or derived from a subject suspected to be suffering from a P. aeruginosa infection and/or at risk of developing a P. aeruginosa infection or being infected by P. aeruginosa. For example, a sample is isolated from or derived from a subject suffering from a disease or disorder selected from the group consisting of a urinary tract infection, a respiratory system infection, dermatitis, a soft tissue infection, bacteremia, a bone infection, a joint infection, a gastrointestinal infection, a burn, a cancer, AIDS and cystic fibrosis.

In one embodiment, the subject is immunosuppressed, immunocompromised or immune deficient.

Preferably the biological sample in which a protein of P. aeruginosa or an antibody that binds thereto is detected is a sample selected from the group consisting of lung, lymphoid tissue associated with the lung, paranasal sinuses, bronchi, a bronchiole, alveolus, ciliated mucosal epithelia of the respiratory tract, mucosal epithelia of the respiratory tract, broncheoalveolar lavage fluid (BAL), alveolar lining fluid, a heart or an extract thereof, a heart valve or an extract thereof, spino-cerebellar fluid, bone faeces, urine, sputum, mucus, saliva, blood, serum, plasma and a PBMC.

In one embodiment a biological sample is obtained previously from a patient.

In one embodiment a biological sample is obtained from a subject by a method selected from the group consisting of surgery or other excision method, aspiration of a body fluid such as hypertonic saline or propylene glycol, broncheoalveolar lavage, bronchoscopy, saliva collection with a glass tube, salivette (Sarstedt AG, Sevelen, Switzerland), Ora-sure (Epitope Technologies Pty Ltd, Melbourne, Victoria, Australia), omni-sal (Saliva Diagnostic Systems, Brooklyn, N.Y., USA) and blood collection using any method known in the art, such as, for example using a syringe.

In another preferred embodiment a biological sample is plasma that has been isolated from blood collected from a patient using a method known in the art.

In one embodiment, a biological sample is treated to lyse a cell in said sample. Such methods include the use of detergents, enzymes, repeatedly freezing and thawing said cells, sonication or vortexing said cells in the presence of glass beads, amongst others.

In another embodiment, a biological sample is treated to denature a protein present in said sample. Methods of denaturing a protein include heating a sample, treatment with 2-mercaptoethanol, or treatment with detergents and other compounds such as, for example, guanidinium or urea.

In yet another embodiment, a biological sample is treated to concentrate a protein is said sample. Methods of concentrating proteins include precipitation, freeze drying, use of funnel tube gels (TerBush and Novick, Journal of Biomolecular Techniques, 10(3); 1999), ultrafiltration or dialysis.

As will be apparent, the diagnostic and prognostic methods provided by the present invention require a degree of quantification to determine either, the amount of a protein that is diagnostic or prognostic of an infection or disease. Such quantification can be determined by the inclusion of appropriate reference samples in the assays described herein, wherein said reference samples are derived from healthy or normal individuals.

In one embodiment, the reference sample comprises a biological sample (for example a cell, tissue, plasma, serum, whole blood, sputum, saliva, or BAL fluid) derived from the same subject when the individual was not suffering from an infection or exacerbation. In another embodiment, the reference sample comprises a biological sample (e.g., a cell, tissue, plasma, serum, whole blood, sputum, saliva, or BAL fluid) derived from a normal healthy individual.

Accordingly, a reference sample and a test (or patient) sample are both processed, analysed or assayed and data obtained for a reference sample and a test sample are compared. In one embodiment, a reference sample and a test sample are processed, analysed or assayed at the same time. In another embodiment, a reference sample and a test sample are processed, analysed or assayed at a different time.

In an alternate embodiment, a reference sample is not included in an assay. Instead, a reference sample is derived from an established data set that has been previously generated. Accordingly, in one embodiment, a reference sample comprises data from a sample population study of healthy individuals, such as, for example, statistically significant data for the healthy range of the integer being tested. Data derived from processing, analysing or assaying a test sample is then compared to data obtained for the sample population.

Data obtained from a sufficiently large number of reference samples so as to be representative of a population allows the generation of a data set for determining the average level of a particular parameter. Accordingly, the amount of a protein that is diagnostic or prognostic of an infection or exacerbation can be determined for any population of individuals, and for any sample derived from said individual, for subsequent comparison to levels of the expression product determined for a sample being assayed. Where such normalized data sets are relied upon, internal controls are preferably included in each assay conducted to control for variation.

Diagnostic Assay Kits P A further aspect of the present invention provides a kit for detecting P. aeruginosa infection in a biological sample. In one embodiment, the kit comprises:
(i) one or more isolated antibodies that bind to a protein of P. aeruginosa or an immunogenic fragment or epitope thereof; and
(ii) means for detecting the formation of an antigen-antibody complex.

In an alternative embodiment, the kit comprises:
(i) an isolated or recombinant protein of P. aeruginosa or an immunogenic fragment or epitope thereof; and
(ii) means for detecting the formation of an antigen-antibody complex.

Optionally, the kit further comprises means for the detection of the binding of an antibody, fragment thereof or a ligand to a protein associated with anaerobic growth of P. aeruginosa. Such means include a reporter molecule such as, for example, an enzyme (such as horseradish peroxidase or alkaline phosphatase), a substrate, a cofactor, an inhibitor, a dye, a radionucleotide, a luminescent group, a fluorescent group, biotin or a colloidal particle, such as colloidal gold or selenium. Preferably such a reporter molecule is directly linked to the antibody or ligand.

In yet another embodiment, a kit additionally comprises a reference sample. Such a reference sample may for example, be a protein sample derived from a biological sample isolated from one or more subjects suffering from a P. aeruginosa infection. Alternatively, a reference sample may comprise a biological sample isolated from one or more normal healthy individuals. Such a reference sample is optionally included in a kit for a diagnostic or prognostic assay.

In another embodiment, a reference sample comprises a peptide that is detected by an antibody or a ligand. Preferably, the peptide is of known concentration. Such a peptide is of use as a standard, for example, various known concentrations of such a peptide may be detected using a prognostic or diagnostic assay described herein.

In yet another embodiment, a kit optionally comprises means for sample preparations, such as, for example, a means for cell lysis.

In yet another embodiment, a kit comprises means for protein isolation (Scopes (In: Protein Purification: Principles and Practice, Third Edition, Springer Verlag, 1994).

### Diagnosis/Prognosis of an Acute Clinical Exacerbation

As an infection by P. aeruginosa is often associated with or causative of an acute clinical exacerbation in a subject suffering from CF, the present invention additionally provides methods for diagnosing, prognosing and/or monitoring an acute clinical exacerbation in a CF subject.

For example, the present invention provides a method for diagnosing an acute pulmonary exacerbation in a subject suffering from cystic fibrosis (CF) or determining a CF subject at risk of developing an acute pulmonary exacerbation, said method comprising diagnosing an infection by P. aeruginosa in the subject by performing a method described herein wherein diagnosis of the infection indicates that the subject is suffering from an acute pulmonary exacerbation or a is at risk of developing an acute pulmonary exacerbation.

In one embodiment, the invention provides a method for determining the response of a subject having cystic fibrosis (CF) and suffering from an acute pulmonary exacerbation to treatment with a therapeutic compound for said exacerbation, said method comprising determining the response of a subject having an infection by P. aeruginosa to treatment with a therapeutic compound for said infection by performing the method described herein, wherein indication that the subject is not responding to said treatment for said infection or has not been rendered free of disease or infection indicates that the subject is not responding to treatment for said exacerbation and/or is not recovering from said exacerbation.

In another embodiment, the present invention provides a method for determining the response of a subject having cystic fibrosis (CF) and suffering from an acute pulmonary exacerbation to treatment with a therapeutic compound for said exacerbation, said method comprising determining the response of a subject having an infection by P. aeruginosa to treatment with a therapeutic compound for said infection by performing the method described herein, wherein indication that the subject is has responded to or is responding to said treatment for said infection or has been rendered free of disease or infection indicates that the subject is responding to or has responded to treatment for said exacerbation and/or is recovering from said exacerbation.

### Prophylactic and Therapeutic Method

A protein of P. aeruginosa or an immunogenic fragment or epitope thereof induces the specific production of an antibody in a subject infected with P. aeruginosa.

Accordingly, the invention additionally provides a method of eliciting the production of antibody against P. aeruginosa comprising administering an isolated or recombinant protein of P. aeruginosa or an immunogenic fragment or epitope thereof to said subject for a time and under conditions sufficient to elicit the production of antibodies, such as, for example, neutralizing antibodies against P. aeruginosa.

Preferably, the neutralizing antibodies are high titer neutralizing antibodies.

The effective amount of the protein of P. aeruginosa or epitope to produce antibodies varies upon the nature of the immunogenic B cell epitope, the route of administration, the animal used for immunization, and the nature of the antibody sought. All such variables are empirically determined by art-recognized means.

The protein of P. aeruginosa or fragment thereof comprising an epitope is readily synthesized using standard techniques, such as the Merrifield method of synthesis (Merrifield, J Am Chem Soc, 85,:2149-2154, 1963) and the myriad of available improvements on that technology (see e.g., Synthetic Peptides: A User's Guide, Grant, ed. (1992) W.H. Freeman & Co., New York, pp. 382; Jones (1994) The Chemical Synthesis of Peptides, Clarendon Press, Oxford, pp. 230.); Barany, G. and Merrifield, R. B. (1979) in The Peptides (Gross, E. and Meienhofer, J. eds.), vol. 2, pp. 1-284, Academic Press, New York; Wunsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474.d/

As is known in the art, synthetic peptides can be produced with additional hydrophilic N-terminal and/or C-terminal amino acids added to the sequence of a fragment or B-cell epitope derived from the full-length protein from P. aeruginosa, such as, for example, to facilitate synthesis or improve peptide solubility. Glycine and/or serine residues are particularly preferred for this purpose.

The peptides of the invention are readily modified for diagnostic purposes, for example, by addition of a natural or synthetic hapten, an antibiotic, hormone, steroid, nucleoside, nucleotide, nucleic acid, an enzyme, enzyme substrate, an enzyme inhibitor, biotin, avidin, streptavidin, polyethylene glycol, a peptidic polypeptide moiety (e.g. tuftsin, polylysine), a fluorescence marker (e.g. FITC, RITC, dansyl, luminol or coumarin), a bioluminescence marker, a spin label, an alkaloid, biogenic amine, vitamin, toxin (e.g. digoxin, phalloidin, amanitin, tetrodotoxin), or a complex-forming agent.

In another embodiment, a protein of P. aeruginosa or a fragment thereof is produced as a recombinant protein.

For expressing protein by recombinant means, a protein-encoding nucleotide sequence is placed in operable connection with a promoter or other regulatory sequence capable of regulating expression in a cell-free system or cellular system. In one embodiment of the invention, nucleic acid comprising a sequence that encodes a protein of P. aeruginosa or an epitope thereof in operable connection with a suitable promoter sequence, is expressed in a suitable cell for a time and under conditions sufficient for expression to occur. Nucleic acid encoding the protein of P. aeruginosa is readily derived from a publicly available amino acid sequence.

In another embodiment, a protein of P. aeruginosa is produced as a recombinant fusion protein, such as for example, to aid in extraction and purification. To produce a fusion polypeptide, the open reading frames are covalently linked in the same reading frame, such as, for example, using standard cloning procedures as described by Ausubel et al. (Current Protocols in Molecular Biology, Wiley Interscience, ISBN 047150338, 1992), and expressed under control of a promoter. Examples of fusion protein partners include glutathione-S-transferase (GST), FLAG (Asp-Tyr-Lys-Asp-Asp-Asp-Asp-Lys), hexahistidine, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the immune function of the protein from P. aeruginosa.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid molecule to which it is operably connected, and which encodes the polypeptide or peptide fragment (i.e., a protein of P. aeruginosa). Preferred promoters can contain additional copies of one or more specific regulatory elements to further enhance expression and/or to alter the spatial expression and/or temporal expression of the said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control.

The prerequisite for producing intact polypeptides and peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L}, or λ_{R} promoters, T7 promoter or the IPTG-inducible tac promoter. A number of other vector systems for expressing the nucleic acid molecule of the invention in E. coli are well-known in the art and are described, for example, in Ausubel et at (In: Current Protocols in Molecular Biology. Wiley Interscience, ISBN 047150338, 1987) or Sambrook et at (In: Molecular cloning, A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (eg. 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing a secreted form of a protein from P. aeruginosa or a derivative thereof in 293T cells, wherein the expressed peptide or protein can be purified free of conspecific proteins, using standard affinity techniques that employ a Nickel column to bind the protein via the His tag.

A wide range of additional host/vector systems suitable for expressing a Group TB protein or an immunological derivative thereof are available publicly, and described, for example, in Sambrook et at (In: Molecular cloning, A laboratory manual, second edition, Cold Spring Harbor Laboratory, Cold Spring Harbor, N.Y., 1989).

Means for introducing the isolated nucleic acid molecule or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

Proteins of the invention can be produced in an isolated form, preferably substantially free of conspecific protein. Antibodies and other affinity ligands are preferred for producing isolated protein. Preferably, the protein will be in a preparation wherein more than about 90% (e.g. 95%, 98% or 99%) of the protein in the preparation is a protein of P. aeruginosa or an epitope thereof.

In a preferred embodiment, the invention provides a method of inducing immunity against P. aeruginosa in a subject comprising administering to said subject an isolated or recombinant protein of P. aeruginosa or immunogenic fragment or epitope thereof for a time and under conditions sufficient to elicit a humoral immune response against said an isolated or recombinant protein of P. aeruginosa or immunogenic fragment or epitope. For example, a protein selected from the group consisting of HitA, PAPS, thioredoxin, GroES, NDK and DNA binding protein HU or mixtures thereof is administered to the subject.

The immunizing antigen may be administered in the form of any convenient formulation as described herein.

By “humoral immune response” means that a secondary immune response is generated against the immunizing antigen sufficient to prevent infection by P. aeruginosa.

Preferably, the humoral immunity generated includes eliciting in the subject a sustained level of antibodies against a B cell epitope in the immunizing antigen. By a “sustained level of antibodies” is meant a sufficient level of circulating antibodies against the B cell epitope to prevent infection by P. aeruginosa.

Preferably, antibodies levels are sustained for at least about six months or 9 months or 12 months or 2 years.

In an alternative embodiment, the present invention provides a method of enhancing the immune system of a subject comprising administering an immunologically active protein of P. aeruginosa or an epitope thereof or a vaccine composition comprising said protein of P. aeruginosa or epitope for a time and under conditions sufficient to confer or enhance resistance against P. aeruginosa in said subject.

By “confer or enhance resistance” is meant that a P. aeruginosa-specific immune response occurs in said subject, said response being selected from the group consisting of:
(i) an antibody against a protein of P. aeruginosa or an epitope of said protein is produced in said subject;
(ii) neutralizing antibodies against P. aeruginosa are produced in said subject;
(iii) a cytotoxic T lymphocyte (CTL) and/or a CTL precursor that is specific for a protein of P. aeruginosa is activated in the subject; and
(iv) the subject has enhanced immunity to a subsequent P. aeruginosa infection.

In a related embodiment this aspect of the invention relates to a method for providing or enhancing immunity against P. aeruginosa in an uninfected human subject comprising administering to said subject an immunologically active protein of P. aeruginosa or an epitope thereof or a vaccine composition comprising said protein of P. aeruginosa or epitope for a time and under conditions sufficient to provide immunological memory against a future infection by P. aeruginosa.

A further aspect of the present invention provides a method of treatment of P. aeruginosa infection in a subject comprising performing a diagnostic method or prognostic method as described herein.

In one embodiment, the present invention provides a method of prophylaxis comprising:
(iii) detecting the presence of P. aeruginosa infection in a biological sample from a subject; and
(iv) administering a therapeutically effective amount of a pharmaceutical composition to reduce the number of pathogenic bacterium in the lung, blood or lymph system of the subject.

### Vaccine Formulations

The present invention clearly contemplates the use of a protein of P. aeruginosa or an immunogenic fragment or epitope thereof in the preparation of a therapeutic or prophylactic subunit vaccine against P. aeruginosa infection in a human or other animal subject.

Accordingly, a further aspect of the invention provides a vaccine comprising a protein of P. aeruginosa or an immunogenic fragment or epitope thereof in combination with a pharmaceutically acceptable diluent.

The protein of P. aeruginosa or immunogenic fragment or epitope thereof is conveniently formulated in a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the pharmaceutical composition are adjusted according to routine skills in the art.

In certain situations, it may also be desirable to formulate the protein of P. aeruginosa or immunogenic fragment or epitope thereof with an adjuvant to enhance the immune response to the B cell epitope. Again, this is strictly not essential. Such adjuvants include all acceptable immunostimulatory compounds such as, for example, a cytokine, toxin, or synthetic composition. Exemplary adjuvants include IL-1, IL-2, BCG, aluminum hydroxide, N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP) 1983A, referred to as MTP-PE), lipid A, MPL and RIBI, which contains three components extracted from bacteria, monophosphoryl lipid A, trehalose dimycolate and cell wall skeleton (MPL+TDM+CWS) in a 2% squalene/Tween 80 emulsion.

It may also be desirable to co-administer biologic response modifiers (BRM) with the protein of P. aeruginosa or immunogenic fragment or epitope thereof, to down regulate suppressor T cell activity. Exemplary BRM's include, but are not limited to, Cimetidine (CIM; 1200 mg/d) (Smith/Kline, PA, USA); Indomethacin (IND; 150 mg/d) (Lederle, N.J., USA); or low-dose Cyclophosphamide (CYP; 75, 150 or 300 mg/m.sup.2) (Johnson/Mead, NJ, USA).

Preferred vehicles for administration of the protein of P. aeruginosa or immunogenic fragment or epitope thereof include liposomes. Liposomes are microscopic vesicles that consist of one or more lipid bilayers surrounding aqueous compartments. (Bakker-Woudenberg et al., Eur. J. Clin. Microbiol. Infect. Dis. 12(Suppl. 1), S61 (1993); and Kim, Drugs 46, 618 (1993)). Liposomes are similar in composition to cellular membranes and as a result, liposomes generally are administered safely and are biodegradable.

Techniques for preparation of liposomes and the formulation (e.g., encapsulation) of various molecules, including peptides and oligonucleotides, with liposomes are well known to the skilled artisan.

Depending on the method of preparation, liposomes may be unilamellar or multilamellar, and can vary in size with diameters ranging from 0.02 .μm to greater than 10 μm. A variety of agents are encapsulated in liposomes. Hydrophobic agents partition in the bilayers and hydrophilic agents partition within the inner aqueous space(s) (Machy et al., LIPOSOMES IN CELL BIOLOGY AND PHARMACOLOGY (John Libbey 1987), and Ostro et al., American J. Hosp. Pharm. 46, 1576 (1989)).

Liposomes can also adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446, 368 (1985)). In the present context, the protein of P. aeruginosa or immunogenic fragment or epitope thereof may be localized on the surface of the liposome, to facilitate antigen presentation without disruption of the liposome or endocytosis. Irrespective of the mechanism or delivery, however, the result is the intracellular disposition of the associated protein of P. aeruginosa or immunogenic fragment or epitope thereof.

Liposomal vectors may be anionic or cationic. Anionic liposomal vectors include pH sensitive liposomes which disrupt or fuse with the endosomal membrane following endocytosis and endosome acidification. Cationic liposomes are preferred for mediating mammalian cell transfection in vitro, or general delivery of nucleic acids, but are used for delivery of other therapeutics, such as peptides or lipopeptides.

Cationic liposome preparations are made by conventional methodologies (Feigner et al, Proc. Nat'l Acad. Sci. USA 84, 7413 (1987); Schreier, Liposome Res. 2, 145 (1992)). Commercial preparations, such as Lipofectin (Life Technologies, Inc., Gaithersburg, Md. USA), are readily available. The amount of liposomes to be administered are optimized based on a dose response curve. Feigner et al., supra.

Other suitable liposomes that are useful in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. (See COLLOIDAL DRUG DELIVERY SYSTEMS, vol. 66, J. Kreuter, ed., Marcel Dekker, Inc. 1994).

Other forms of delivery particle, for example, microspheres and the like, also are contemplated for delivery of the protein of P. aeruginosa or immunogenic fragment or epitope thereof.

Guidance in preparing suitable formulations and pharmaceutically effective vehicles, are found, for example, in REMINGTON'S PHARMACEUTICAL SCIENCES, chapters 83-92, pages 1519-1714 (Mack Publishing Company 1990) (Remington's), which are hereby incorporated by reference.

Alternatively, the peptide or derivative or variant is formulated as a cellular vaccine via the administration of an autologous or allogeneic antigen presenting cell (APC) or a dendritic cell that has been treated in vitro so as to present the peptide on its surface.

Nucleic acid-based vaccines that comprise nucleic acid, such as, for example, DNA or RNA, encoding the immunologically active protein of P. aeruginosa or epitope(s) and cloned into a suitable vector (e.g. vaccinia, canary pox, adenovirus, or other eukaryotic virus vector) are also contemplated. Preferably, DNA encoding a associated with anaerobic growth of P. aeruginosa protein is formulated into a DNA vaccine, such as, for example, in combination with the existing Calmette-Guerin (BCG) or an immune adjuvant such as vaccinia virus, Freund's adjuvant or another immune stimulant.

The present invention is further described with reference to the following non-limiting examples.

### Example 1

### Determining levels of CF-Specific Antibody Repertoires

### 1.1 Biological Samples

Clinical whole blood CF samples were collected and the crude plasma used for the capture column were combined from four exacerbated CF adults in the age group 22- to 37-years old. Predicted FEV₁ values were between 22-65% and the subjects have had 2-4 exacerbations in the last 12 months. Microbiological testing was performed on collected sputum samples. All adult CF subjects used had profuse P. aeruginosa growth in the lungs. In addition, one CF adult also had pulmonary S. aureus infection.

1.2 Preparation of Protein from P. aeruginosa

Overnight cultures of P. aeruginosa PA01 (200 mL) were pelleted by centrifugation (20 minutes at 4000 g, room temperature). The precipitated cells were washed twice in water and resuspended in Lysis Buffer A (50 mM Tris-HCl pH 7.6, 0.1 mM EDTA, 20% sucrose)+protease inhibitors (1× Complete Protease Inhibitor Cocktail, Roche Diagnostics, Basel, Switzerland). Cells were lysed using a Branson sonifier, model 250-450, using 70% of maximal amplitude for 4×10 seconds and unbroken cells were pelleted by centrifugation (4000 g, 10 min, 4° C.). Another sonification step was performed on the pellet, whereafter the two supernatants were pooled and proteins isolated by acetone precipitation. Precipitation proteins were resolubilised in 10 mM PBS pH 7.2

Membrane proteins: membrane proteins were extracted using the ProteoPrep membrane extraction kit essentially as recommended by manufacturer (Proteome Systems, Woburn, US). However, the resulting pellet after the last 50 mM Tris-HCl, pH 7.3 wash was resuspended in 10 mM PBS pH 7.4 containing 1% Triton-X, 15 mM Tris-HCl pH 7.5 and 20 mM DTT. After solubilisation, sample was incubated with 60 mM iodoacetamide for 2 hours at room temperature. The two protein extracts were pooled prior further use.

### 1.3 2D Gel Electrophoresis

Eleven centimetres pH 3-10 IPG strips were purchased from Amersham (Uppsala, Sweden). Isoelectric focusing was conducted as per manufacturer's instructions using an IsoElectrIQ² unit from Proteome Systems (Woburn, Mass.). Second dimension 6-15% Tris-Acetate Gelchip gels were run as recommended by manufacturer (Proteome Systems, Woburn, Mass.). Arrayed proteins were transferred to PVDF-P membranes (Millipore, Billerica, Mass.) by using semi-wet membrane-blotting cassettes accompanying the IsoElectrIQ² unit from Proteome Systems (Woburn, US).

### 1.4 Immunoprofiling

Circulating antibodies found in crude plasma of CF subjects or healthy controls were probed against 2DE arrayed proteins extracted from an overnight culture of P. aeruginosa. Membranes were probed according to standard western blotting procedure (Sambrook et al. Eds., In: Molecular Cloning: A laboratory manual. (Cold Spring Harbor Laboratory Press, Cold Spring Harbor, N.Y., 2001). Approximately, 1.6-2.0 ng/ml crude plasma IgG was used in the immuno-fingerprinting experiments and resulting immuno complexes were detected by chemiluminescence using a HRP-conjugated antihuman IgG antibody according to standard procedures (Sambrook et al)

### 1.5 Results

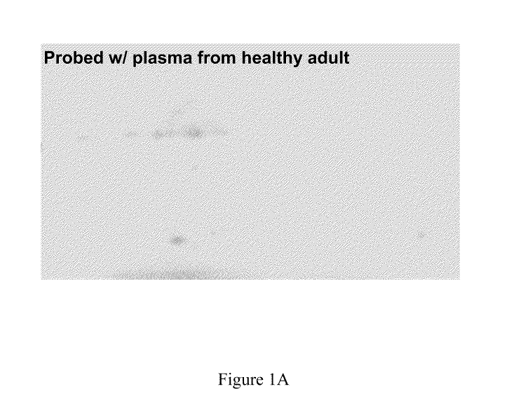

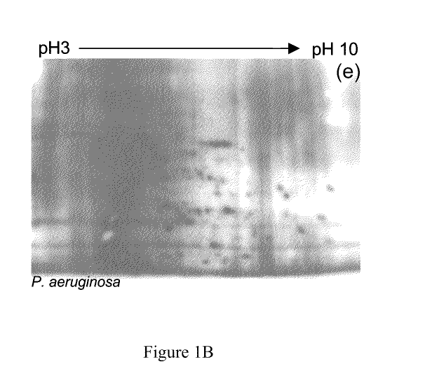

As shown in FIG. 1B CF subjects suffering from a P. aeruginosa infection produce antibodies capable of binding to cytosolic or membrane P. aeruginosa proteins. In contrast, healthy non-CF control subjects were not immunoreactive toward 2-DE arrayed P. aeruginosa proteins (FIG. 1A). These data show that a humoral immune response can be exploited as a tool to specifically identify putative biomarker candidates.

### Example 2

### Isolation and Identification of CF-Specific Immuno-Reactive Pathogenic Proteins

Subjects that suffer from cystic fibrosis are prone to infections by P. aeruginosa. As shown in Example 1, P. aeruginosa infected CF subjects raise antibodies to proteins expressed by the infecting bacterium. To identify proteins from P. aeruginosa that may be useful in diagnosing such an infection, immunoglobulin fraction was isolated from CF subjects and used to isolate immunogenic proteins expressed by the infectious bacterium.

### 2.1 Preparation of an Immunocapture Column

An immuno-capture column was generated from a total of 5 mL pooled crude plasma from five exacerbated CF patients (total protein concentration of ˜40 mg/mL). IgG was bound to Protein G sepharose by incubating the pooled plasma with 10 mL 50% slurry of Protein G sepharose. The matrix was washed in 10 mM PBS pH 7.4 and bound IgG was irreversibly immobilised utilizing DSS. The generated column is referred to as the capture column.

2.2 Capture of Immunogenic Protein from P. aeruginosa

The capture column was incubated overnight with the native P. aeruginosa protein extract at 4° C. at constant rotation and beads were subsequently harvested by centrifugation. The flow-through was collected and saved for subsequent incubation steps (the protein extract was passed over the capture column three times in each capture). The harvested beads were washed 3 times in 10 mM PBS pH 7.4 and captured proteins were eluted with 50 mM glycine pH 2.7. The column was extensively washed with first 50 mM glycine pH 2.7 then 10 mM PBS pH 7.2 prior subsequent incubation steps.

Eluted proteins were precipitated and subsequently resolubilised in Cellular and Organelle Membrane solubilizing reagent from the ProteoPrep Universal Extraction kit (Sigma, St. Louis, Mo.). Following the instruction in the ProteoPrep kit the solubilized proteins were reduced and alkylated with a final concentration of 5 mM tri-n-butylphosphine and 10 mM acrylamide, respectively.

### 2.3 Two-Dimensional Gel Electrophoresis

Eleven centimetre pH 3-10 IPG strips were purchased from Amersham (Uppsala, Sweden). Isoelectric focusing was conducted as per manufacturer's instructions using an IsoElectrIQ² unit from Proteome Systems (Woburn, Mass.). Second dimension 6-15% Tris-Acetate Gelchip gels were run as recommended by manufacturer (Proteome Systems, Woburn, Mass.). Arrayed proteins were visualised by silver-staining (Shevchenko et al., Mass spectrometric sequencing of proteins silver-stained polyacrylamide gels. 68, 850-858. 1996).

### 2.4 MS Analysis

Protein spots of interest were excised and prepared for MS analysis as described in Katayama et al (Improvement of in-gel digestion protocol for peptide mass fingerprinting by matrix-assisted laser desorption/ionization time-of-flight mass spectrometry) and Kussmann et al. Peptides were eluted with ˜1.5 μl MALDI matrix solution (70% ACN, 0.1% TFA, 1.5 mg/ml alpha-cyano-4-hydroxycinnamic acid (Sigma, St. Louis, Mo.). Peptide mass fingerprints (PMF) were generated by matrix-assisted laser desorption/ionisation-time-of-flight-mass spectrometry (MALDI-TOF-MS) using an Axima CFR (Kratos, Manchester, UK) or an ABI MALDI MS/MS (AME Bioscience, London, UK).

### 2.5 Results

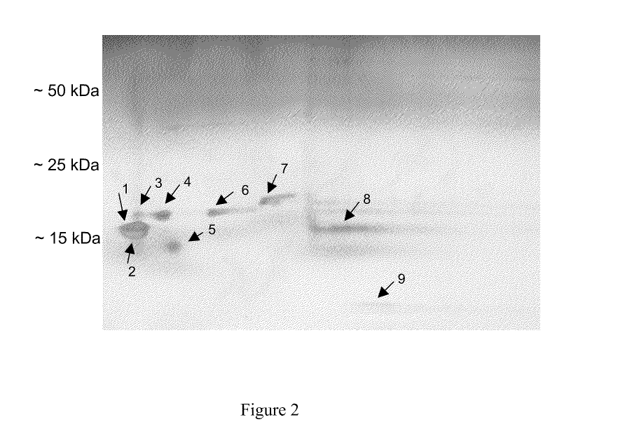

As shown in FIG. 2, nine immunogenic proteins were captured from P. aeruginosa protein extracts using the immuno capture column. These proteins were analysed using MALDI MS and MALDI MS/MS and their identity confirmed as being P. aeruginosa derived proteins Results are summarized in Table 1. Table 2 shows the actual peptides identified by peptide mass fingerprinting.

TABLE 1
Antigenic P. aeruginosa proteins identified using immunoglobulin from a CF
subject.

| | | Swissprot | | % | | | |
|---|---|---|---|---|---|---|---|
| | Protein | accession | Matching | sequence | | | SEQ ID |
| Spot no. | identification | no. | peptides | coverage | TpI | TMW | NO: |
| 1 | Ferric iron- | Q9HVA8 | 4 | 16% | 5.5 | 36 | 1 |
| | binding | | | | | | |
| | periplasmic | | | | | | |
| | protein HitA | | | | | | |
| 2 | Thioredoxin | O05927 | 4 | 14% | 6.02 | 30 | 2 |
| | dependent | | | | | | |
| | PAPS | | | | | | |
| 3 | Thioredoxin | Q9X2T1 | 6 | 56% | 4.7 | 12 | 3 |
| 4 | Thioredoxin | Q9X2T1 | 7 | 67% | 4.7 | 12 | 3 |
| 5 | | | | | | | |
| 6 | GroES | P30720 | 9 | 74% | 5.2 | 10 | 4 |
| 7 | Nucleoside | Q59636 | 6 | 69% | 5.5 | 16 | 5 |
| | diphosphate | | | | | | |
| | kinase (NDK) | | | | | | |
| 8 | | | | | | | |
| 9 | DNA-binding | P05384 | 6 | 64% | 9.7 | 10 | 6 |
| | protein HU - | | | | | | |
| | beta | | | | | | |

TpI; theoretical pI,
TMW; theoretical molecular weight

TABLE 2
Peptides identified usina mass spectrometry

| | | Matching | | |
|---|---|---|---|---|
| Spot | Protein | peptides/ | Aa sequence of | SEQ ID |
| no. | identification | % coverage | matching peptides² | NO: |
| 1 | Ferric iron- | 4/16 | AFQDKTGIQVK | 7 |
| | binding | | GQEEAEDWLTGLK (confirmed by PSD) | 8 |
| | periplasmic | | AILSQSAEYPMRK | 9 |
| | protein HitA | | LKGQEEAEDWLTGLK | 10 |
| 2 | Thioredoxin | 4/14 | DGHGECCGIR (confirmed by PSD) | 11 |
| | dependent | | MLPFATIPATER | 12 |
| | PAPS | | MLPFATIPATER MSO modified | 13 |
| | | | EHYGIAIDVLSPDPR | 14 |
| | | | LAGVRAWATGQR | 15 |
| 3 | Thioredoxin | 6/56 | GIPTLMLFK | 16 |
| | | | GIPTLMLFK MSO modified | 17 |
| | | | MIAPVLDEVAR | 18 |
| | | | MIAPVLDEVARDYQGK | 19 |
| | | | LNIDENQDTPPKYGVR | 20 |
| | | | DYQGKLK | 21 |
| | | | MIAPVLDEVAR MSO modified | 22 |
| | | | SEHIVNVTDASFEQDVLK | 23 |
| 4 | Thioredoxin | 7/67 | DYQGKLK | 24 |
| | | | GIPTLMLFK | 25 |
| | | | GIPTLMLFK MSO modified | 26 |
| | | | MIAPVLDEVAR | 27 |
| | | | MIAPVLDEVARDYQGK | 28 |
| | | | SEHIVNVTDASFEQDVLK | 29 |
| | | | VCKLNIDENQDTPPK | 30 |
| | | | LNIDENQDTPPKYGVR | 31 |
| | | | SQLAAFLDANI | 32 |
| 5 | | | | |
| 6 | GroES | 9/74 | LRPLHDR | 33 |
| | | | GEVVAVGTGR | 34 |
| | | | MKLRPLHDR | 35 |
| | | | MKLRPLHDR MSO modified | 36 |
| | | | VVFGPYSGSNAIK | 37 |
| | | | VLDNGEVRALAVK | 38 |
| | | | TAGGIVLPGSAAEKPNR | 39 |
| | | | VGDKVVFGPYSGSNAIK | 40 |
| | | | VLDNGEVR | 41 |
| | | | SEEETKTAGGIVLPGSAAEKPNR | 42 |
| 7 | Nucleoside | 6/69 | NVIGEILTRFEK | 43 |
| | diphosphate | | NVIGEILTR | 44 |
| | kinase (NDK) | | EIAYFFAATEVCER | 45 |
| | | | ADFAVSIDENAVHGSDSEASAAR | 46 |
| | | | DLVSFMTSGPVVVQVLEGEDAIAK | 47 |
| | | | ERPFFK | 48 |
| 8 | | | | |
| 9 | DNA-binding | 6/64 | IAAAKIPGFK | 49 |
| | protein HU- | | TGRNPQTGKPIK | 50 |
| | beta | | SELIDAIAASADIPK | 51 |
| | | | AGDSVVLVGFGTFAVK | 52 |
| | | | AGDSVVLVGFGTFAVKER | 53 |
| | | | MNKSELIDAIAASADIPK | 54 |

### Example 3

### Characterisation of P. aeruginosa NDK

NDK enzymatic activity is regulated by phosphorylation. In fact, phosphorylation of NDK is considered to be important in extracellular alginate synthesis in P. aeruginosa. Alginate synthesis is a dominant virulence factor of P. aeruginosa. Accordingly, studies were undertaken to identify a phosphorylation site in P. aeruginosa NDK.

### 3.1 Phosphoprotein Characterisation

Tryptic digests of phosphoproteins were incubated with 5U alkaline phosphatase (Roche Applied Science, Indianapolis, US) as described by Stensballe et al., Proteomics. 1: 207-22, 2001. Peptides were purified from half of the treated sample and eluted onto MALDI target plates as described in Example 2. PMF data was acquired on an AXIMA CFR (Kratos, Manchester, UK). Amino acid sequence confirmation was obtained by post-source decay using an Axima CFR (Kratos, Manchester, UK), but the dephosphorylated sample was sulfonated prior PSD MALDI analysis to optimise for y-ion collection Wang, et al., Rapid Commun. Mass Spectrom. 18: 96-102, 2004.

### 3.2 Results

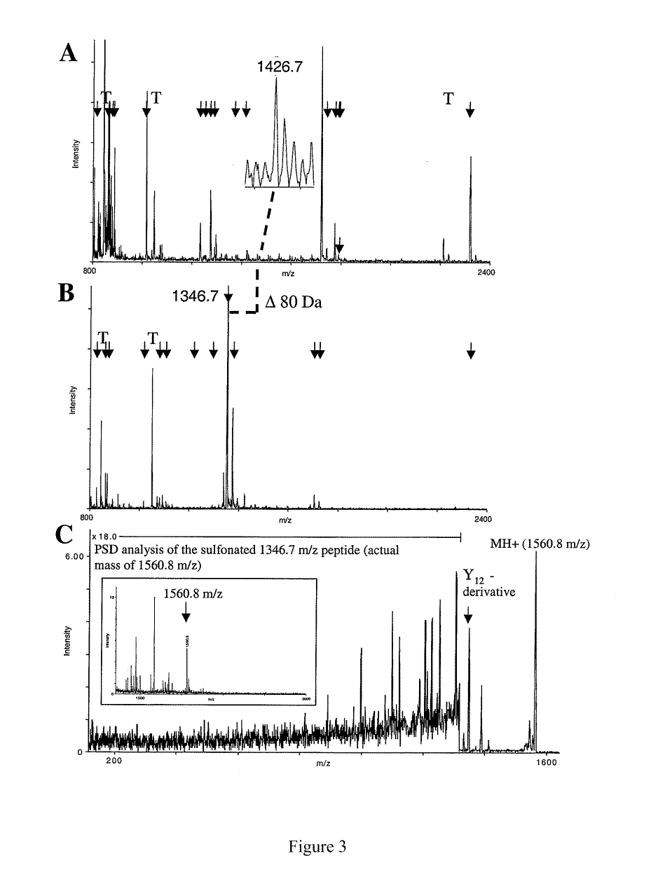

The MS-based identification of P. aeruginosa encoded NDK (FIG. 3A) was further characterised by treating an aliquot of tryptic digested NDK with alkaline phosphatase to identify any phosphorylated peptides. A loss of a phosphate group is reflected by a decrease in peptide mass of 80 Da. MS analysis of the phosphatase-treated tryptic digests showed a dominant 1346.7 m/z peptides (FIG. 3A), matching the theoretically oxidated tryptic peptide from amino acid residues 34 to 45 of SEQ ID NO: 5 (i.e. VVAAKM_{oxidated}VQLSER). The peptide mass of 1426.7 obtained in the initial MS analysis of non-phosphatase treated tryptic digested NDK is likely to be the cognate peptide (FIG. 3A). The phosphatase treated tryptic digest of NDK was sulfonated to optimise AXIMA-based post-source decay (PSD) fragmentation analysis, resulting in modified peptide masses of +214 Da. PSD analysis of a peptide mass of 1560.8 m/z (sulfonated cognate of 1346.7 m/z) confirmed that the 1346.4 m/z peptide was indeed the VVAAKM_{oxidated}VQLSER (SEQ ID NO: 52) peptide of NDK (FIG. 3C). These data suggested that the immunocaptured P. aeruginosa encoded NDK protein was at least phosphorylated at serine residue 43 (i.e. of SEQ ID NO: 5).

### Example 4

### Use of an identified P. aeruginosa Protein to Determine a Subject Suffering from a P. aeruginosa Infection

Aliquots from four of the identified P. aeruginosa proteins were excised from the 2-DE array described in Example 2, washed in H₂O and 1 mM DTT. Proteins were extracted by two successive overnight incubations in 0.1% SDS, 50 mM Tris-HCl pH 7.9, 0.1 mM EDTA, 150 mM NaCl and 5 mM DTT at 4° C. by vigorous shaking, precipitated and resolubilised in 50 Tl PBS. Only 6 Tl of the extracted proteins were applied to nitrocellulous membrane strips (Biorad, Hercules, Calif., US). Membranes were blocked with 5% (w/v) skim milk in 10 mM Tris-HCl, 100 mM NaCl and 0.2% Tween-20 pH 9.0 prior to use. Anchors were applied to membranes for subsequent localisation of antigenic targets. Crude plasma from healthy controls and CF subjects were diluted 1:3 in PBST buffer (10 mM PBS, 0.05% (v/v) Tween-20) containing 0.5% (w/v) skim milk, and filtered through a 0.22-Tm PVDF membrane (Millipore). A chemical printer, ChIP™, (Proteome Systems Ltd., Sydney, Australia and Shimadzu, Biotech, Kyoto, Japan) was used to dispense five applications of 0.15 TL 1:3 plasma aliquots onto the immobilised pathogenic proteins, PBS and 100 ng BSA. Grid arrays containing 4- or 5-spot positions, where each spot position represented one patient sample, were printed onto targets of membranes. X- and Y-coordinates were established using the software ImagepIQ™ version 1.0 (Proteome Systems Ltd., Sydney, Australia). Approximately 100 TL PBST was used to wash away excess plasma proteins. Bound antibody was detected by printing 0.1 Tl HRP-conjugated rabbit anti-human IgG, 1:50000 in PSBT-M buffer (Chemicon Australia Pty., Victoria, Australia). Chemiluminescence was then detected using a standard procedure (Sambrook et al, supra). The size of the printed grid array depended on the area of the immobilised antigenic target, which in current study had a diameter of ˜5 mm.

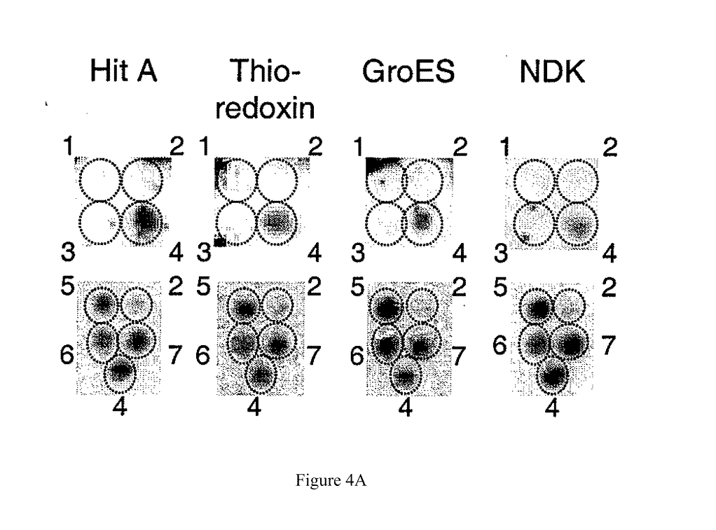

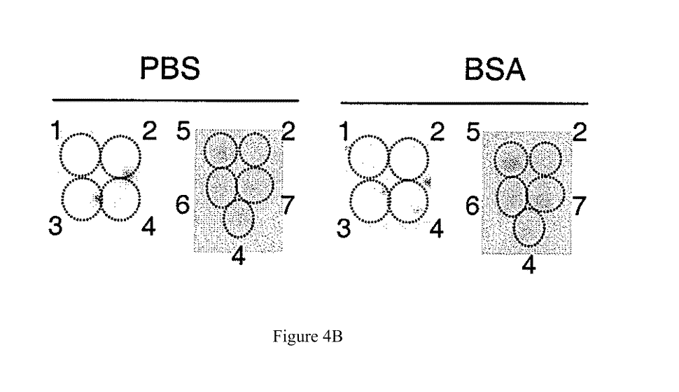

Serological immunoreactivities of up to five patients were simultaneously determined towards P. aeruginosa HitA, thioredoxin, GroES and NDK using a chemical printer, ChIP™. As shown in FIG. 4A all screened CF subjects were immunoreactive towards the pathogenic proteins, in contrast to the serological non-reactive healthy controls, hence supporting clinically relevant expression of these pathogen-encoded proteins in CF subjects. Furthermore, negative controls (BSA or PBS) indicate that the antibody responses were specific for the proteins tested (FIG. 4B).

1. A method for diagnosing an infection by Pseudomonas aeruginosa in a subject comprising detecting in a biological sample from said subject an immunogenic protein of P. aeruginosa or modified forma immunogenic fragment or epitope thereof, wherein the presence of said immunogenic protein, modified form, immunogenic fragment or epitope in the sample is indicative of infection.

2. The method according to claim 1 wherein the protein of P. aeruginosa is a protein associated with anaerobic growth of P. aeruginosa.

3. The method according to claim 1 wherein the protein of P. aeruginosa is a stress response protein of P. aeruginosa.

4. The method according to claim 1 wherein the protein of P. aeruginosa is associated with or involved in extracellular alginate production by P. aeruginosa.

5. The method according to claim 1 wherein the protein of P. aeruginosa is selected from the group consisting of ferric iron-binding protein (HitA), thioredoxin dependent reductase (PAPS), thioredoxin, heat shock protein GroES, nucleotide dependent kinase (NDK), DNA-binding protein HU and mixtures thereof.

6. (canceled)

7. The method according to claim 1 wherein the modified form of a protein of P. aeruginosa is a phosphorylated protein or a glycosylated protein or a lipidated protein or a fucosylated protein or a cleaved protein or a degraded protein.

8. The method according to claim 7 wherein the modified form of a protein of P. aeruginosa is a phosphorylated nucleotide dependent kinase (NDK).

9. (canceled)

10. The method according to claim 1 wherein said method comprises contacting a biological sample derived from the subject with one or more antibodies or ligands capable of binding to a protein of P. aeruginosa or an immunogenic fragment or epitope thereof, and detecting the formation of an antigen-antibody/ligand complex.

11. The method according to claim 10 wherein an antibody is a polyclonal antibody.

12. The method according to claim 10 wherein the antibody is a monoclonal antibody.

13. The method according to claim 1 wherein the subject suffers from a disease or disorder selected from the group consisting of a urinary tract infection, a respiratory system infection, dermatitis, a soft tissue infection, bacteremia, a bone infection, a joint infection, a gastrointestinal infection, a burn, a cancer, AIDS and cystic fibrosis.

14. The method according to claim 1 wherein the subject is immunosuppressed, immunocompromised or immune deficient.

15. The method according to claim 1 wherein the sample is selected from the group consisting of sputum, serum, plasma, whole blood, saliva, urine, pleural fluid and mixtures thereof.

16. The method according to claim 1 wherein the sample is derived from a body fluid selected from the group consisting of sputum, serum, plasma, whole blood, saliva, urine, pleural fluid and mixtures thereof.

17. A method for diagnosing an infection by Pseudomonas aeruginosa in a subject comprising detecting in a biological sample from said subject an antibody that binds to a protein of P. aeruginosa or a modified form, immunogenic fragment or epitope thereof, wherein said protein, modified form, immunogenic fragment or epitope generates an antibody response in the subject and wherein the presence of said antibody in the sample is indicative of infection and/or exacerbation.

18. The method according to claim 17 wherein the protein of P. aeruginosa is a protein associated with anaerobic growth of P. aeruginosa.

19. The method according to claim 17 wherein the protein of P. aeruginosa is a stress response protein.

20. The method according to claim 17 wherein the protein of P. aeruginosa is associated with or involved in extracellular alginate production by P. aeruginosa.

21. The method according to claim 17 wherein the protein of P. aeruginosa is selected from the group consisting of ferric iron-binding protein (HitA), thioredoxin dependent reductase (PAPS), thioredoxin, heat shock protein GroES, nucleotide dependent kinase (NDK), DNA-binding protein HU and mixtures thereof.

22. (canceled)

23. The method according to claim 17 wherein the modified form of a protein of P. aeruginosa is a phosphorylated protein or a glycosylated protein or a lipidated protein or a fucosylated protein or a cleaved protein or a degraded protein.

24. The method according to claim 17 wherein the modified form of a protein of P. aeruginosa is a phosphorylated nucleotide dependent kinase (NDK).

25. (canceled)

26. The method according to claim 17 wherein said method comprises contacting a biological sample derived from the subject with a protein of P. aeruginosa or an immunogenic fragment or epitope thereof for a time and under conditions sufficient for an antibody-antigen complex to form, and detecting the formation of an antigen-antibody complex.

27. The method according to claim 17 wherein the subject suffers from a disease or disorder selected from the group consisting of a urinary tract infection, a respiratory system infection, dermatitis, a soft tissue infection, bacteremia, a bone infection, a joint infection, a gastrointestinal infection, a burn, a cancer, AIDS and cystic fibrosis.

28. The method according to claim 17 wherein the subject is immunosuppressed, immunocompromised or immune deficient.

29. The method according to claim 17 wherein the sample is selected from the group consisting of serum, plasma, whole blood, pleural fluid and mixtures thereof.

30. The method according to claim 17 wherein the sample is derived from a body fluid selected from the group consisting of serum, plasma, whole blood, pleural fluid and mixtures thereof.

31. A process for determining the response of a subject having an infection by Pseudomonas aeruginosa to treatment with a therapeutic compound for said infection, said process comprising performing the method of claim 1 to thereby detect a protein of P. aeruginosa or a modified form, immunogenic fragment or epitope thereof in a biological sample from said subject, wherein a level of the protein or fragment or epitope that is enhanced compared to the level of that protein or fragment or epitope detectable in a normal or healthy subject indicates that the subject is not responding to said treatment or has not been rendered free of disease or infection and wherein a level of the protein or fragment or epitope that is lower than the level of the protein or fragment or epitope detectable in a subject suffering from said infection by P. aeruginosa indicates that the subject is responding to said treatment or has been rendered free of disease or infection.

32-62. (canceled)

63. The method of claim 1, comprising contacting a biological sample derived from the subject with one or more antibodies capable of binding to ferric iron-binding protein (HitA) or an immunogenic fragment or epitope thereof for a time and under conditions sufficient for an antibody-antigen complex to form, and detecting the antigen-antibody complex.

64. The method of claim 1, comprising contacting a biological sample derived from the subject with one or more antibodies capable of binding to thioredoxin dependent reductase (PAPS) or an immunogenic fragment or epitope thereof for a time and under conditions sufficient for an antibody-antigen complex to form, and detecting the antigen-antibody complex.

65. The method of claim 1, comprising contacting a biological sample derived from the subject with one or more antibodies capable of binding to thioredoxin or an immunogenic fragment or epitope thereof for a time and under conditions sufficient for an antibody-antigen complex to form, and detecting the antigen-antibody complex.

66. The method of claim 1, comprising contacting a biological sample derived from the subject with one or more antibodies capable of binding to heat shock protein GroES or an immunogenic fragment or epitope thereof for a time and under conditions sufficient for an antibody-antigen complex to form, and detecting the antigen-antibody complex.

67. The method of claim 1, comprising contacting a biological sample derived from the subject with one or more antibodies capable of binding to nucleotide dependent kinase (NDK) or an immunogenic fragment or epitope thereof for a time and under conditions sufficient for an antibody-antigen complex to form, and detecting the antigen-antibody complex.

68. The method of claim 1, comprising contacting a biological sample derived from the subject with one or more antibodies capable of binding to DNA-binding protein HU or an immunogenic fragment or epitope thereof for a time and under conditions sufficient for an antibody-antigen complex to form, and detecting the antigen-antibody complex.

69. The method of claim 17, comprising contacting a biological sample derived from the subject comprising antibodies with ferric iron-binding protein (HitA) or an immunogenic fragment or epitope thereof for a time and under conditions sufficient for an antibody-antigen complex to form, and detecting the antigen-antibody complex.

70. The method of claim 17, comprising contacting a biological sample derived from the subject comprising antibodies with a thioredoxin dependent reductase (PAPS) or an immunogenic fragment or epitope thereof for a time and under conditions sufficient for an antibody-antigen complex to form and detecting the antigen-antibody complex.

71. The method of claim 17, comprising contacting a biological sample derived from the subject comprising antibodies with a thioredoxin or an immunogenic fragment or epitope thereof for a time and under conditions sufficient for and antibody-antigen complex to form and detecting the antigen-antibody complex.

72. The method of claim 17, comprising contacting a biological sample derived from the subject comprising antibodies with a heat shock protein GroES or an immunogenic fragment or epitope thereof for a time and under conditions sufficient for an antibody-antigen complex to form and detecting the antigen-antibody complex.

73. The method of claim 17, comprising contacting a biological sample derived from the subject comprising antibodies with a nucleotide dependent kinase (NDK) or an immunogenic fragment or epitope thereof for a time and under conditions sufficient for an antibody-antigen complex to form and detecting the antigen-antibody complex.

74. The method of claim 17, comprising contacting a biological sample derived from the subject comprising antibodies with a DNA-binding protein HU or an immunogenic fragment or epitope thereof, for a time and under conditions sufficient for an antibody-antigen complex to form and detecting the antigen-antibody complex.

75. A method for diagnosing an acute pulmonary exacerbation in a subject suffering from cystic fibrosis (CF) or determining a CF subject at risk of developing an acute pulmonary exacerbation, said method comprising diagnosing an infection by P. aeruginosa in the subject by performing a method according to claim 1 wherein diagnosis of the infection indicates that the subject is suffering from an acute pulmonary exacerbation or a is at risk of developing an acute pulmonary exacerbation.

76. A method for determining the response of a subject having cystic fibrosis (CF) and suffering from an acute pulmonary exacerbation to treatment with a therapeutic compound for said exacerbation, said method comprising determining the response of a subject having an infection by P. aeruginosa to treatment with a therapeutic compound for said infection by performing the method according to claim 31, wherein, indication that the subject is not responding to said treatment for said infection or has not been rendered free of disease or infection indicates that the subject is not responding to treatment for said exacerbation and/or is not recovering from said exacerbation.

77. A method for determining the response of a subject having cystic fibrosis (CF) and suffering from an acute pulmonary exacerbation to treatment with a therapeutic compound for said exacerbation, said method comprising determining the response of a subject having an infection by P. aeruginosa to treatment with a therapeutic compound for said infection by performing the method according to claim 31, wherein, indication that the subject is has responded to or is responding to said treatment for said infection or has been rendered free of disease or infection indicates that the subject is responding to or has responded to treatment for said exacerbation and/or is recovering from said exacerbation.

78. A method of preparing a reagent for diagnosing an infection by P. aeruginosa and/or an acute clinical exacerbation, said method comprising obtaining one or more antibodies against a protein selected from the group consisting of ferric iron-binding protein (HitA), thioredoxin dependent reductase (PAPS), thioredoxin, heat shock protein GroES, nucleotide dependent kinase (NDK) and DNA-binding protein HU.

79. A method of preparing a reagent for diagnosing an infection by P. aeruginosa and/or an acute clinical exacerbation, said method comprising obtaining one or more proteins selected from the group consisting of ferric iron-binding protein (HitA), thioredoxin dependent reductase (PAPS), thioredoxin, heat shock protein GroES, nucleotide dependent kinase (NDK) and DNA-binding protein HU.

80. A method of treatment of an infection by P. aeruginosa in a subject or an acute pulmonary exacerbation in a subject suffering from cystic fibrosis, said method comprising:
(i) performing the method of claim 1 to thereby detect the presence of P. aeruginosa infection in a biological sample from a subject; and
(ii) administering a therapeutically effective amount of a pharmaceutical composition to reduce the number of pathogenic bacterium in the lung, blood or lymph system of the subject.

81. A method for eliciting the production of an antibody against P. aeruginosa in a subject comprising administering an isolated immunogenic protein of P. aeruginosa or an immunogenic fragment or epitope thereof to said subject for a time and under conditions sufficient to elicit the production of antibodies against the immunogenic protein, fragment or epitope.

82. The method according to claim 81 wherein the antibody is a neutralizing antibody against P. aeruginosa.

83. The method according to claim 81 wherein the protein of P. aeruginosa is a protein associated with anaerobic growth of P. aeruginosa.

84. The method according to claim 81 wherein the protein of P. aeruginosa is a stress response protein of P. aeruginosa.

85. The method according to claim 81 wherein the protein of P. aeruginosa is associated with or involved in extracellular alginate production by P. aeruginosa.

86. The method according to claim 81 wherein the protein of P. aeruginosa is selected from the group consisting of ferric iron-binding protein (HitA), thioredoxin dependent reductase (PAPS), thioredoxin, heat shock protein GroES, nucleotide dependent kinase (NDK), DNA-binding protein HU and mixtures thereof.

87.-88. (canceled)

89. A vaccine comprising an immunogenic protein of P. aeruginosa or an immunogenic fragment or epitope thereof in combination with a pharmaceutically acceptable diluent.

90. A kit for detecting P. aeruginosa infection in a biological sample, said kit comprising:
(i) one or more isolated antibodies that bind to an immunogenic protein of P. aeruginosa selected from the group consisting of ferric iron-binding protein (HitA), thioredoxin dependent reductase (PAPS), thioredoxin, heat shock protein GroES, nucleotide dependent kinase (NDK), DNA-binding protein HU or an immunogenic fragment or epitope thereof; and
(ii) means for detecting the formation of an antigen-antibody complex.

91. A kit for detecting P. aeruginosa infection in a biological sample, said kit comprising:
(i) one or more isolated immunogenic proteins of P. aeruginosa selected from the group consisting of ferric iron-binding protein (HitA), thioredoxin dependent reductase (PAPS), thioredoxin, heat shock protein GroES, nucleotide dependent kinase (NDK), DNA-binding protein HU or an immunogenic fragment or epitope thereof; and
(ii) means for detecting the formation of an antigen-antibody complex.

92. A method of treatment of an infection by P. aeruginosa in a subject or an acute pulmonary exacerbation in a subject suffering from cystic fibrosis, said method comprising:
(i) performing the method of claim 17 to thereby detecting the presence of P. aeruginosa infection in a biological sample from a subject; and
(ii) administering a therapeutically effective amount of a pharmaceutical composition to reduce the number of pathogenic bacterium in the lung, blood or lymph system of the subject.


SEQUENCE LISTING (54 sequences)

SEQ ID NO: 1 - protein, 335 aa, pseudomonas aeruginosa
MPVRLLNRFRHAALACLLGSLSLNAAADPVTLTLYNGQHAATGIAIAKAFQDKTGIQVKIRKGGDGQLASQITEEGARSPADVLYTEESPPLIRLASAGLLAKLEPETLALVEPEHAGGNGDWIGITARTRVLAYNPKKIDEKDLPKSLMDLSDPSWSGRFGFVPTSGAFLEQVAAVIKLKGQEEAEDWLTGLKAFGSIYTNNVTAMKAVENGEVDMALINNYYWYTLKKEKGELNSRLHYFGNQDPGALVTVSGAAVLKSSKHPREAQQFVAFMLSEEGQKAILSQSAEYPMRKGMQADPALKPFAELDPPKLTPADLGEASEALSLERDVGLN

SEQ ID NO: 2 - protein, 267 aa, pseudomonas aeruginosa
MLPFATIPATERNSAAQHQDPSPMSQPFDLPALASSLADKSPQDILKAAFEHFGDELWISFSGAEDVVLVDMAWKLNRNVKVFSLDTGRLHPETYRFIDQVREHYGIAIDVLSPDPRLLEPLVKEKGLFSFYRDGHGECCGIRKIEPLKRKLAGVRAWATGQRRDQSPGTRSQVAVLEIDGAFSTPEKPLYKFNPLSSMTSEEVWGYIRMLELPYNSLHERGYISIGCEPCTRPVLPNQHEREGRWWWEEATHKECGLHAGNLISKA

SEQ ID NO: 3 - protein, 108 aa, pseudomonas aeruginosa
MSEHIVNVTDASFEQDVLKADGPVLVDYWAEWCGPCKMIAPVLDEVARDYQGKLKVCKLNIDENQDTPPKYGVRGIPTLMLFKDGNVEATKVGALSKSQLAAFLDANI

SEQ ID NO: 4 - protein, 97 aa, pseudomonas aeruginosa
MKLRPLHDRVVIRRSEEETKTAGGIVLPGSAAEKPNRGEVVAVGTGRVLDNGEVRALAVKVGDKVVFGPYSGSNAIKVDGEELLVMGESEILAVLED

SEQ ID NO: 5 - protein, 143 aa, pseudomonas aeruginosa
MALQRTLSIIKPDAVSKNVIGEILTRFEKAGLRVVAAKMVQLSEREAGGFYAEHKERPFFKDLVSFMTSGPVVVQVLEGEDAIAKNRELMGATDPKKADAGTIRADFAVSIDENAVHGSDSEASAAREIAYFFAATEVCERIR

SEQ ID NO: 6 - protein, 90 aa, pseudomonas aeruginosa
MNKSELIDAIAASADIPKAVAGRALDAVIESVTGALKAGDSVVLVGFGTFAVKERAARTGRNPQTGKPIKIAAAKIPGFKAGKALKDAVN

SEQ ID NO: 7 - protein, 11 aa, artificial sequence
  fragment of P. aeruginosa HitA protein
AFQDKTGIQVK

SEQ ID NO: 8 - protein, 13 aa, artificial sequence
  fragment of P. aeruginosa HitA protein
GQEEAEDWLTGLK

SEQ ID NO: 9 - protein, 13 aa, artificial sequence
  fragment of P. aeruginosa HitA protein
AILSQSAEYPMRK

SEQ ID NO: 10 - protein, 15 aa, artificial sequence
  fragment of P. aeruginosa HitA protein
LKGQEEAEDWLTGLK

SEQ ID NO: 11 - protein, 10 aa, artificial sequence
  fragment of P. aeruginosa PAPS protein
DGHGECCGIR

SEQ ID NO: 12 - protein, 12 aa, artificial sequence
  fragment of P. aeruginosa PAPS protein
MLPFATIPATER

SEQ ID NO: 13 - protein, 12 aa, artificial sequence
  fragment of P. aeruginosa PAPS protein
MLPFATIPATER

SEQ ID NO: 14 - protein, 15 aa, artificial sequence
  fragment of P. aeruginosa PAPS protein
EHYGIAIDVLSPDPR

SEQ ID NO: 15 - protein, 12 aa, artificial sequence
  fragment of P. aeruginosa PAPS protein
LAGVRAWATGQR

SEQ ID NO: 16 - protein, 9 aa, artificial sequence
  fragment of P. aeruginosa thioredoxin protein
GIPTLMLFK

SEQ ID NO: 17 - protein, 9 aa, artificial sequence
  fragment of P. aeruginosa thioredoxin protein
GIPTLMLFK

SEQ ID NO: 18 - protein, 11 aa, artificial sequence
  fragment of P. aeruginosa thioredoxin protein
MIAPVLDEVAR

SEQ ID NO: 19 - protein, 16 aa, artificial sequence
  fragment of P. aeruginosa thioredoxin protein
MIAPVLDEVARDYQGK

SEQ ID NO: 20 - protein, 16 aa, artificial sequence
  fragment of P. aeruginosa thioredoxin protein
LNIDENQDTPPKYGVR

SEQ ID NO: 21 - protein, 7 aa, artificial sequence
  fragment of P. aeruginosa thioredoxin protein
DYQGKLK

SEQ ID NO: 22 - protein, 11 aa, artificial sequence
  fragment of P. aeruginosa thioredoxin protein
MIAPVLDEVAR

SEQ ID NO: 23 - protein, 18 aa, artificial sequence
  fragment of P. aeruginosa thioredoxin protein
SEHIVNVTDASFEQDVLK

SEQ ID NO: 24 - protein, 7 aa, artificial sequence
  fragment of P. aeruginosa thioredoxin protein
DYQGKLK

SEQ ID NO: 25 - protein, 9 aa, artificial sequence
  fragment of P. aeruginosa thioredoxin protein
GIPTLMLFK

SEQ ID NO: 26 - protein, 9 aa, artificial sequence
  fragment of P. aeruginosa thioredoxin protein
GIPTLMLFK

SEQ ID NO: 27 - protein, 11 aa, artificial sequence
  fragment of P. aeruginosa thioredoxin protein
MIAPVLDEVAR

SEQ ID NO: 28 - protein, 16 aa, artificial sequence
  fragment of P. aeruginosa thioredoxin protein
MIAPVLDEVARDYQGK

SEQ ID NO: 29 - protein, 18 aa, artificial sequence
  fragment of P. aeruginosa thioredoxin protein
SEHIVNVTDASFEQDVLK

SEQ ID NO: 30 - protein, 15 aa, artificial sequence
  fragment of P. aeruginosa thioredoxin protein
VCKLNIDENQDTPPK

SEQ ID NO: 31 - protein, 16 aa, artificial sequence
  fragment of P. aeruginosa thioredoxin protein
LNIDENQDTPPKYGVR

SEQ ID NO: 32 - protein, 11 aa, artificial sequence
  fragment of P. aeruginosa thioredoxin protein
SQLAAFLDANI

SEQ ID NO: 33 - protein, 7 aa, artificial sequence
  fragment of P. aeruginosa GroES protein
LRPLHDR

SEQ ID NO: 34 - protein, 10 aa, artificial sequence
  fragment of P. aeruginosa GroES protein
GEVVAVGTGR

SEQ ID NO: 35 - protein, 9 aa, artificial sequence
  fragment of P. aeruginosa GroES protein
MKLRPLHDR

SEQ ID NO: 36 - protein, 9 aa, artificial sequence
  fragment of P. aeruginosa GroES protein
MKLRPLHDR

SEQ ID NO: 37 - protein, 13 aa, artificial sequence
  fragment of P. aeruginosa GroES protein
VVFGPYSGSNAIK

SEQ ID NO: 38 - protein, 13 aa, artificial sequence
  fragment of P. aeruginosa GroES protein
VLDNGEVRALAVK

SEQ ID NO: 39 - protein, 17 aa, artificial sequence
  fragment of P. aeruginosa GroES protein
TAGGIVLPGSAAEKPNR

SEQ ID NO: 40 - protein, 17 aa, artificial sequence
  fragment of P. aeruginosa GroES protein
VGDKVVFGPYSGSNAIK

SEQ ID NO: 41 - protein, 8 aa, artificial sequence
  fragment of P. aeruginosa GroES protein
VLDNGEVR

SEQ ID NO: 42 - protein, 23 aa, artificial sequence
  fragment of P. aeruginosa GroES protein
SEEETKTAGGIVLPGSAAEKPNR

SEQ ID NO: 43 - protein, 12 aa, artificial sequence
  fragment of P. aeruginosa NDK protein
NVIGEILTRFEK

SEQ ID NO: 44 - protein, 9 aa, artificial sequence
  fragment of P. aeruginosa NDK protein
NVIGEILTR

SEQ ID NO: 45 - protein, 14 aa, artificial sequence
  fragment of P. aeruginosa NDK protein
EIAYFFAATEVCER

SEQ ID NO: 46 - protein, 23 aa, artificial sequence
  fragment of P. aeruginosa NDK protein
ADFAVSIDENAVHGSDSEASAAR

SEQ ID NO: 47 - protein, 24 aa, artificial sequence
  fragment of P. aeruginosa NDK protein
DLVSFMTSGPVVVQVLEGEDAIAK

SEQ ID NO: 48 - protein, 6 aa, artificial sequence
  fragment of P. aeruginosa NDK protein
ERPFFK

SEQ ID NO: 49 - protein, 10 aa, artificial sequence
  fragment of P. aeruginosa HU-beta DNA-binding protein
IAAAKIPGFK

SEQ ID NO: 50 - protein, 12 aa, artificial sequence
  fragment of P. aeruginosa HU-beta DNA-binding protein
TGRNPQTGKPIK

SEQ ID NO: 51 - protein, 15 aa, artificial sequence
  fragment of P. aeruginosa HU-beta DNA-binding protein
SELIDAIAASADIPK

SEQ ID NO: 52 - protein, 16 aa, artificial sequence
  fragment of P. aeruginosa HU-beta DNA-binding protein
AGDSVVLVGFGTFAVK

SEQ ID NO: 53 - protein, 18 aa, artificial sequence
  fragment of P. aeruginosa HU-beta DNA-binding protein
AGDSVVLVGFGTFAVKER

SEQ ID NO: 54 - protein, 18 aa, artificial sequence
  fragment of P. aeruginosa HU-beta DNA-binding protein
MNKSELIDAIAASADIPK